(19) United States
(12) Patent Application Publication
(10) Pub. No.: US 2026/0061063 A1
(43) Pub. Date: Mar. 5, 2026

(54) RNA-BASED THERAPEUTIC DELIVERY WITH OXIDATIVE STRESS-RESPONSIVE COACERVATES

(71) Applicant: Clemson University Research Foundation, Clemson, SC (US)

(72) Inventors: Jessica M. Larsen, Pendleton, SC (US); Chloe Forenzo, Glen Gardner, NJ (US)

(21) Appl. No.: 19/315,991

(22) Filed: Sep. 2, 2025

Related U.S. Application Data
(60) Provisional application No. 63/689,004, filed on Aug. 30, 2024.

(51) Int. Cl. A61K 47/59; A61K 47/60

(52) U.S. Cl. CPC A61K 47/595; A61K 47/60

(57) ABSTRACT

Disclosed herein are novel and advanced technique for the modification of complex coacervates to induce structural changes releasing ribonucleic acid (RNA)-based molecules in response to oxidative stress. This approach leverages the reactivity of reactive oxygen species (ROS) generated during oxidative stress to trigger structural changes in the modified cationic coacervate component that induces phase miscibility and subsequently releases RNA molecules from the coacervate matrix.

### CROSS-REFERENCE TO RELATED APPLICATION(S)

This application claims the benefit under 35 U.S.C. § 119(e) to U.S. Provisional Application 63/689,004, filed Aug. 30, 2024 and entitled “RNA-Based Therapeutic Delivery with Oxidative Stress-Responsive Coacervates,” which is hereby incorporated herein by reference in their entireties.

### GOVERNMENT SUPPORT

This invention was made with government support under Grant No. 2100800 awarded by the U.S. National Science Foundation. The government has certain rights in the invention.

### FIELD

The various embodiments herein relate to novel and advanced techniques of delivering ribonucleic acid (RNA) molecules to cells through the modification of the polycationic component of RNA complex coacervates to induce stimuli-triggered structural changes that release RNA molecules in response to elevated oxidative stress.

### BACKGROUND

The delivery of exogenous ribonucleic acid (RNA) molecules is a versatile therapeutic approach that involves transporting these molecules into the cytoplasm of cells, where they can perform various functions essential for therapeutic outcomes. For instance, messenger RNA (mRNA) undergoes translation in the cytoplasm to produce specific proteins with therapeutic potential, targeting a wide range of conditions, including infectious diseases, genetic disorders, cardiovascular issues, and neurological disorders (see Paunovska, K., Loughrey, D. & Dahlman, J. E. Drug delivery systems for RNA therapeutics. Nat. Rev. Genet. 23, 265-280 (2022)). The revolutionary use of mRNA technology in developing COVID-19 vaccines has transformed the field of vaccinology, offering unparalleled advantages in terms of rapid and adaptable vaccine production (see Nance, K. D. & Meier, J. L. Modifications in an Emergency: The Role of N1-Methylpseudouridine in COVID-19 Vaccines. ACS Cent. Sci. 7, 748-756 (2021)). Beyond mRNA, other RNA types such as small interfering RNA (siRNA), microRNA (miRNA), and short hairpin RNA (shRNA) have significant therapeutic potential due to their ability to regulate gene expression, silence specific genes, or interfere with viral replication. Long non-coding RNA (lncRNA), guide RNA (gRNA) for CRISPR applications, and ribosomal RNA (rRNA) are also being explored for their diverse roles in gene regulation and genome editing (See Kim, Y. RNA Therapy: Current Status and Future Potential. Chonnam Med J. 56, 87-93 (2020)). In contrast to conventional virus-based (viral) methods which entail the laborious processes of collection, culture, inactivation, purification, and shipment, RNA-based therapies, including those utilizing mRNA and other RNA molecules, present a streamlined approach, with steps limited to laboratory synthesis, digital sequencing, and electronic transmission, offering faster and more efficient treatments (See Zeng, C., Zhang, C., Walker, P. G. & Dong, Y. Formulation and Delivery Technologies for mRNA Vaccines. Curr Top Microbiol Immunol 437, 71-110 (2022)). Additionally, RNA-based therapeutics, including mRNA and siRNA, are significantly more cost-effective than many traditional therapies. For example, the COVID-19 mRNA vaccine's cost per treatment ranges between $100-$200, significantly lower than the costs associated with some protein-based therapies, such as the recently FDA-approved gene therapy for sickle cell disease, which can exceed $2 million per treatment (See Martonosi, S., Behzad, B., & Cummings, K. Pricing the COVID-19 vaccine: A mathematical approach. Omega. 103, 102451 (2021) and Quach, D., Jiao, B., Basu, A., Bender, M. A., Hankins, J., Ramsey, S. & Devine, B. A landscape analysis and discussion of value of gene therapies for sickle cell disease. Expert Rev. Pharmacoeconomics Outcomes Res. 6, 891-911 (2022)). Despite the promising potential of RNA delivery, there are significant challenges that need to be addressed to make it more effective and safer. Conventional RNA delivery methods face hurdles such as rapid degradation of RNA molecules by enzymes, inefficient cellular uptake, and poor stability during circulation. Additionally, off-target effects may lead to unintended protein expression, potentially causing adverse effects. While nanoparticle-mediated intracellular delivery overcomes some of these barriers, improving precise targeted delivery while minimizing off-target effects is of utmost importance for successful RNA-based therapies.

Complex coacervation is a phase separation process driven by oppositely charged polyelectrolytes that spontaneously form dense liquid-liquid aggregates (See Forenzo, C. & Larsen, J. Complex Coacervates as a Promising Vehicle for mRNA Delivery: A Comprehensive Review of Recent Advances and Challenges. Mol. Pharm. (2023). doi:10.1021/acs.molpharmaceut.3c00439). These aggregates exhibit responsiveness to alterations in the surrounding physiochemical conditions, such as pH, temperature, and ionic strength (see Blocher, W. C. & Perry, S. L. Complex coacervate-based materials for biomedicine. Wiley Interdiscip. Rev. Nanomedicine Nanobiotechnology 9, 76-78 (2017); Moulik, S. P., Rakshit, A. K., Pan, A. & Naskar, B. An Overview of Coacervates: The Special Disperse State of Amphiphilic and Polymeric Materials in Solution. Colloids and Interfaces 6, (2022); and Chen, S., Guo, Q. & Yu, J. Bio-inspired functional coacervates. Aggregate 3, e293 (2022). Complex coacervates occur naturally in cells and contribute to the creation of membraneless organelles that play crucial roles in various cellular functions, including intracellular signaling, RNA regulation, and compartmentalization of biomolecules (see Boeynaems, S., Holehouse, A. S., Weinhardt, V., Kovacs, D., Van Lindt, J., Larabell, C., Bosch, L. Van Den, Das, R., Tompa, P. S., Pappu, R. V. & Gitler, A. D. Spontaneous driving forces give rise to protein-RNA condensates with coexisting phases and complex material properties. Proc. Nat. Acad. Sci. U.S.A 116, 7889-7898 (2019)). The intrinsic affinity of coacervates for RNA molecules presents a remarkable opportunity to advance therapeutic RNA delivery utilizing coacervate phase separation to release RNA molecules in response to specific cues. Through harnessing reversible charge interactions, these systems hold the potential for encapsulating RNA molecules, ensuring protection and enabling triggerable release. The versatility of coacervates lies in their ability to generate a distinct macromolecular-rich fluid phase in an aqueous environment. This phase separation not only shields encapsulated molecules from degradation while providing a means of triggered release but can also facilitate their uptake by cells (See Li, Y., Humphries, B., Wang, Z., Lang, S., Huang, X., Xiao, H., Jiang, Y. & Yang, C. Complex Coacervation-Integrated Hybrid Nanoparticles Increasing Plasmid DNA Delivery Efficiency in Vivo. ACS Appl. Mater. Interfaces 8, 30735-30746 (2016)). Spermine, a natural polycationic molecule with multiple amine groups, readily coacervates with RNA molecules via electrostatic interactions, stemming from spermine's positive amine groups and RNA's negative phosphate backbone (ss Aumiller, W. M., Pir Cakmak, F., Davis, B. W. & Keating, C. D. RNA-Based Coacervates as a Model for Membraneless Organelles: Formation, Properties, and Interfacial Liposome Assembly. Langmuir 32, 10042-10053 (2016)). These electrostatic interactions between spermine and RNA play a pivotal role in effectively encapsulating and safeguarding the RNA molecules during coacervation, enhancing RNA stability and facilitating controlled release mechanisms (see Marianelli, A. M., Miller, B. M. & Keating, C. D. Impact of macromolecular crowding on RNA/spermine complex coacervation and oligonucleotide compartmentalization. Soft Matter 14, 368-378 (2018)).

Reactive Oxygen Species (ROS) are profoundly reactive oxygen-containing molecules that play pivotal roles in cellular signaling and regulation (see Zhang, J., Wang, X., Vikash, V., Ye, Q., Wu, D., Liu, Y. & Dong, W. ROS and ROS-Mediated Cellular Signaling. Oxid. Med. Cell. Longev. 2016, (2016)). These molecules are inherent byproducts of diverse metabolic processes and are instrumental in upholding cellular equilibrium. Excessive ROS levels can lead to oxidative stress, damage cellular components and contribute to various disorders, including but not limited to infectious diseases and cancers. Additionally, their intracellular presence can also be elevated by surrounding environmental conditions or even artificially induced through exposure to ionizing radiation (IR) (Akbari, A., Jelodar, G., Nazifi, S., Afsar, T. & Nasiri, K. Oxidative Stress as the Underlying Biomechanism of Detrimental Outcomes of Ionizing and Non-Ionizing Radiation on Human Health: Antioxidant Protective Strategies. Zahedan J. Res. Med. Sci. 21, (2019)). Given that over half of all cancer patients undergo radiotherapy, which leverages IR-induced ROS elevation in tumors, harnessing this mechanism for targeted therapeutic delivery presents a promising avenue for enhancing treatment efficacy (Holley, A. K., Miao, L., St. Clair, D. K. & St. Clair, W. H. Redox-modulated phenomena and radiation therapy: The central role of superoxide dismutases. Antioxidants Redox Signal. 20, 1567-1589 (2014)). By designing delivery systems that respond to increased ROS levels, RNA release can be triggered specifically at sites where oxidative stress is elevated. When utilized as a trigger for RNA release from coacervates, this strategy offers a novel approach to controlled delivery for therapeutic purposes, allowing targeted interventions in conditions characterized by heightened ROS levels, including instances of inflammation, specific cancer types, and even the realm of space exploration (See Forenzo, C. & Larsen, J. Bridging Clinical Radiotherapy and Space Radiation Therapeutics through Reactive Oxygen Species (ROS)—Triggered Delivery. Free Radic. Biol. Med. 219, 88-103 (2024). doi:10.1016/j.freeradbiomed.2024.04.219).

There is a need in the art for improved RNA-based therapeutic delivery vehicles with increased specificity to the target and reduced off-target effects.

### BRIEF SUMMARY

Discussed herein are various embodiments to improve RNA-based therapeutic delivery. Embodiments herein include enhancements to the responsiveness of cationic polymers such as spermine to reactive oxygen species (ROS) to formulate ROS-responsive RNA/spermine coacervates as enhanced RNA-based delivery vehicles. Embodiments herein introduce the novel approach of modifying spermine with a disulfide-containing compound 3,3′-dithiobis(sulfosuccinimidyl propionate) (DTSSP) to enable it ROS-responsive. Upon coacervation with RNA molecules, this innovative system leverages ROS triggers to achieve controlled and targeted RNA release, signifying a significant advancement in the realm of RNA-based therapeutic delivery. This invention is applicable to a variety of RNA types, including but not limited to messenger RNA (mRNA), small interfering RNA (siRNA), microRNA (miRNA), long non-coding RNA (lncRNA), short hairpin RNA (shRNA), CRISPR RNA (crRNA), guide RNA (gRNA), ribosomal RNA (rRNA), and transfer RNA (tRNA).

In Example 1 a composition for ribonucleic acid (RNA)-based therapy comprises a complex coacervate of RNA and a cationic polymer.

Example 2 relates to the composition of Example 1 further comprising a nanoparticle encasing the complex coacervate.

Example 3 relates to the composition of Example 1 wherein the cationic polymer is a spermine or a spermidine.

Example 4 relates to the composition of Example 1 wherein the cationic polymer is spermine and is modified with DTSSP.

Example 5 relates to the composition of Example 1 wherein the spermine is modified with DTSSP at a molar ratio between 1:0 and 1:1 spermine/DTSSP.

Example 6 relates to the composition of Example 1 wherein the RNA is modified RNA.

Example 7 relates to the composition of Example 1 wherein the RNA is mRNA, siRNA, miRNA, lncRNA, shRNA, crRNA, gRNA, rRNA, or tRNA.

Example 8 relates to the composition of Example 1 wherein the nanoparticle has a diameter between 100-200 nm.

Example 9 relates to the composition of Example 1 further comprising a stabilizing agent.

Example 10 relates to the composition of Example 1 wherein the stabilizing agent is polyethylene glycol (PEG) or PEG-diamine.

In Example 11 a method of delivering RNA to a cytosol of a cell comprises modifying a cationic composition with DTSSP; adding the cationic composition to an RNA composition to form a cationic polymer/RNA structure; encapsulating the cationic polymer/RNA structure in a nanoparticle; contacting the nanoparticle to a cell containing cytosol, the cell optionally located in an area of heightened reactive oxygen species (ROS) levels; and optionally increasing a reactive oxygen species (ROS) level of the cytosol to modulate a separation of the cationic polymer and the RNA.

Example 12 relates to the method according to Example 11 wherein the cationic polymer is spermine.

Example 13 relates to the method according to Example 11 wherein the ROS level is increased by a disease, inflammation, or ionizing radiation.

Example 14 relates to the method according to Example 11 wherein the nanoparticle has a diameter between 100-200 nm.

Example 15 relates to the method according to Example 11 wherein the RNA is mRNA, siRNA, miRNA, lncRNA, shRNA, crRNA, gRNA, rRNA, or tRNA.

Example 16 relates to the method according to Example 11 wherein the separation is monitored by measuring the light absorbance (turbidity), by measuring fluorescence, or by measuring a radiolabel.

In Example 17 a method of providing RNA therapy to a patient comprises modifying a spermine composition with DTSSP; adding the spermine to a RNA composition to form a spermine/RNA structure; encapsulating the RNA/spermine complex in a nanoparticle; delivering the nanoparticle to a region of therapy in a patient; and optionally exposing the region of therapy to ionizing radiation.

Example 18 relates to the method according to Example 16 wherein the nanoparticle is delivered to the region of therapy via injection or intravascular administration.

Example 19 relates to the method according to Example 16 wherein the nanoparticle has a diameter between 100-200 nm.

Example 20 relates to the method according to Example 16 wherein the RNA is delivered to the cytosol for therapeutic applications.

Example 21 relates to the method according to Example 16 wherein the RNA is labeled.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a graphic depicting the coacervation of RNA into dense liquid droplets (example shown with mRNA). The coacervated phase protects the RNA molecules from degradation, facilitates endocytosis and enables targeted or triggered release.

FIG. 2 is a graphic depicting the steps of coacervation of RNA molecules with spermine (example shown with model RNA polyuridylic acid (polyU)), with a phase contrast microscopy image of the resulting coacervates. RNA inherently coacervates with the polyamine spermine due to the electrostatic interactions between the positively charged amine groups on spermine and the negatively charged phosphate backbone of the RNA. The coacervates can be seen as dense liquid condensates.

FIG. 3 shows the coacervation interactions between RNA and modified spermine (example shown with mRNA), followed by polymersome-mediated intracellular delivery and ROS activation to release mRNA for subsequent translation

FIG. 4 is a depiction of the coacervation process between DTSSP-Modified Spermine (spermine:DTSSP molar ratio of 1:0.25) and Cyanine 5 (Cy5)—Tagged Green Fluorescent Protein (GFP)—encoding mRNA, conveying the potential of the modified spermine to coacervate longer RNAs with more complex sequences and the ability to translate into proteins.

FIG. 5 Is a proposed Mechanism of Spermine Modification (Crosslinking via DTSSP) and Mass Spectrometry analysis of resulting products with various modification ratios. The nucleophilic primary amine groups of spermine participate in crosslinking by attacking the activated ester groups of DTSSP. This process leads to the displacement of NHS groups and the formation of the modified spermine-DTSSP crosslink). The modification enables spermine to be responsive to reactive oxygen species that generate, for example, after biologics are hit by ionizing radiation. Three primary products are formed (X, Y, and Z) dependent on the initial molar reactant ratio of spermine to DTSSP.

FIG. 6 is a 1H NMR spectra demonstrating the reaction between spermine and DTSSP. The top spectrum shows pure DTSSP, while the bottom spectrum shows unmodified spermine. The middle spectrum displays spermine modified with DTSSP at a 1:0.125 molar ratio.

FIG. 7A depicts the Influence of spermine modification on coacervation capability. A. Turbidity data (fitted to eqn (1)) is plotted as a function of wt % spermine for a 0.05 wt % polyuridylic acid (polyU) solution. The comparison is made between unmodified spermine (squares) and spermine modified with 1:0.125 (diamonds), 1:0.25 (triangles), and 1:0.5 (circles) molar ratios of spermine:DTSSP (3,3′-Dithiobis(sulfosuccinimidylpropionate). Phase contrast microscope images of the 1:0.25 modified coacervates are shown at each measured weight percent to provide insights into the morphological changes induced by varying the modified spermine concentration, shedding light on the coacervation process at a microscopic level (scale bar=130 μm).

FIG. 7B shows phase contrast microscopy images of unmodified and each extent of modified spermine-based coacervates at 0.02 wt % spermine and 0.05 wt % polyU (scale bar=130 μm).

FIG. 8 illustrates the impact of polyethylene glycol (PEG) derivates on 0.05 wt % polyuridylic acid (polyU) and 0.02 wt % spermine coacervate stability. Normalized turbidity measurements at A is 15 minutes and C is at 24 hours for unmodified (bars 1-3) and coacervates modified with various molar ratios of spermine:DTSSP (3,3′-Dithiobis(sulfosuccinimidylpropionate), (1:0.125—bars 4-6, 1:0.25—bars 7-9, & 1:0.5—bars 10-12) formulated with or without 0.1 wt % PEG (6 k) (diagonal stripes) or PEG-diamine (6 kDa) (checkers). Both stabilizers reduce degradation across all modification levels and time points. Corresponding phase contrast images at B is 15 minutes and D is 24 hours show that coacervates lacking stabilizers exhibit fusion, flattening, or incomplete droplet formation, while the addition of PEG or PEG-diamine promotes the formation and maintenance of spherical, well-defined droplets (scale bar=130 μm). These results demonstrate that both PEG-based excipients confer sustained coacervate stabilization, independent of spermine modification extent. Normalized turbidity changes between groups at each time point were compared using two-way ANOVA. *p<0.05; **p<0.01, ***p<0.001, ****p<0.0001.

FIG. 9 shows the phase contrast microscopy of 0.05 wt % polyuridylic acid (polyU) and 0.02 wt % spermine coacervates at 15 minutes with and without stabilizers (scale bar=130 μm). Modification extents include various molar ratios of spermine:DTSSP (3,3′-Dithiobis(sulfosuccinimidylpropionate), and stabilizers include either 0.1 wt % polyethylene glycol (PEG) (6 k) or PEG-diamine (6 kDa). In the absence of stabilizers, droplets show signs of fusion, flattening, or incomplete formation. Both PEG and PEG-diamine promote the formation of well-defined, spherical coacervates across all modification levels.

FIG. 10 shows the phase contrast microscopy of 0.05 wt % polyuridylic acid (polyU) and 0.02 wt % spermine coacervates at 24 hours with and without stabilizers (scale bar=130 μm). Samples were incubated with moderate shaking at 37° C., with or without 0.1 wt % polyethylene glycol (PEG) (6 k) or PEG-diamine (6 k). Modification extents include various molar ratios of spermine:DTSSP (3,3′-Dithiobis(sulfosuccinimidylpropionate). Coacervates treated with either stabilizer maintain their morphology and droplet density over time, while unstabilized samples exhibit greater droplet fusion and loss of structure.

FIG. 11 shows the effect of polyethylene glycol (PEG)—diamine (6 k) concentration on 0.05 wt % polyuridylic acid (polyU) and 0.02 wt % spermine coacervate morphology after 24 hours (scale bar=130 μm). Samples were incubated with moderate shaking at 37° C., with between 0.1 wt % and 1.0 wt % PEG-diamine conditions. Modification extents include various molar ratios of spermine:DTSSP (3,3′-Dithiobis(sulfosuccinimidylpropionate). Increasing PEG-diamine concentration does not significantly alter droplet morphology or density, indicating that the stabilizing effect is maintained across this concentration range.

FIG. 12 shows redox-responsivity of 0.05 wt % polyuridylic acid (polyU) and 0.02 wt % spermine coacervates upon hydrogen peroxide exposure. A. Rate of turbidity change [% turbidity/min] of modified coacervates after 10-minute incubation with hydrogen peroxide, normalized to the rate observed in unmodified coacervates. Each series represents a different extent of spermine modification classified by molar ratio of spermine:DTSSP (3,3′-Dithiobis(sulfosuccinimidylpropionate): 1:0.125 (first bar in each set), 1:0.25 (second), and 1:0.5 (third bar). Increased DTSSP modification and higher hydrogen peroxide concentrations result in greater rates of coacervate destabilization, demonstrating redox-responsivity. B. Representative phase contrast microscopy images of unmodified and modified coacervates after 10 minutes of exposure to hydrogen peroxide (scale bar=340 μm). Images show morphology changes and loss of coacervate integrity with increasing modification extent and oxidative stress, supporting the conclusion that the disulfide-containing crosslink drives redox-induced disassembly. Normalized rates between groups at each hydrogen peroxide concentration were compared using two-way ANOVA. *p<0.05; **p<0.01, ***p<0.001.

FIG. 13 shows the effect of PEG and PEG-diamine stabilization on redox-responsivity of 0.05 wt % polyuridylic acid (polyU) and 0.02 wt % spermine coacervates. A. Rate of turbidity change in response to 1.67 M hydrogen peroxide for coacervates for 10 minutes with no stabilizer, 0.1 wt % PEG-diamine (checkers) or 0.1 wt % PEG (diagonal stripes), normalized to unmodified coacervates for each modification extent (1:0.125—first bar in each set, 1:0.25—second bar, 1:0.50—third bar, molar ratio of spermine:DTSSP (3,3′-Dithiobis(sulfosuccinimidylpropionate))). PEG-diamine preserves partial redox-responsivity, while PEG alone blocks coacervate disassembly. B. Phase contrast microscopy images of unmodified and 1:0.50 modified coacervates after hydrogen peroxide exposure for 10 minutes show droplet destabilization in the no stabilizer and PEG-diamine conditions of modified coacervates, but not in any unmodified or PEG-stabilized coacervates (scale bar=130 μm). Normalized rates between groups were compared using two-way ANOVA. *p<0.05; ***p<0.001, ns: not significant.

FIG. 14 shows the phase contrast images of 0.05 wt % polyuridylic acid (polyU) and 0.02 wt % spermine coacervates incubated with 1.67 M hydrogen peroxide for 10 minutes, in the presence of no stabilizer, 0.1 wt % polyethylene glycol (PEG)—diamine (6 k), or 0.1 wt % PEG (6 k). Modification extents include various molar ratios of spermine:DTSSP (3,3′-Dithiobis(sulfosuccinimidylpropionate). Across all modification extents, PEG-stabilized coacervates retain droplet morphology, while both no-stabilizer and PEG-diamine-stabilized samples show varying degrees of disassembly. At lower modification levels, PEG-diamine retains partial destabilization, though less pronounced than at higher extents, supporting a mechanism of electrostatically mediated redox-responsivity in modified PEG-diamine systems.

FIG. 15 shows contrasting turbidity changes in coacervates following radiation exposure with modified and unmodified spermine. Coacervates containing 0.05 wt % polyU and either 0.01 wt % unmodified spermine (black) or 0.02 wt % modified spermine (red) underwent either five- or ten-minute ionizing radiation exposure at a dose rate of 0.1422 mGy/min, resulting in cumulative absorbed doses of 0.711 mGy and 1.422 mGy respectively. Percent turbidities were normalized to pre-irradiation measurements for each sample. A large drop in turbidity indicates structural changes causing phase miscibility and loss of coacervate structure, conveying ROS-triggered RNA release.

FIG. 16 illustrates a schematic of a water/oil/water double emulsion microfluidic capillary device used to generate and characterize polymer-based nanoparticles (polymersomes) that allow ample phase separation for coacervate encapsulation during polymersome formation. Contrast staining enhances the visualization of the polymersomes through transmission electron microscopy.

FIG. 17 is a ROS-triggered release of fluorescently labeled RNA from redox-responsive coacervates. Coacervates were formed by mixing spermine and polyU in the presence of 2% AlexaFluor568-labeled 20-mer polyU to visualize RNA localization. Phase contrast, Texas Red fluorescence (AlexaFluor568), and overlay images are shown for: (1) free RNA (no spermine), (2) unmodified spermine/polyU coacervates, and (3) 1:0.5 DTSSP-modified spermine/polyU coacervates, both before and after treatment with 1.67 M H₂O₂ for 15 minutes.

FIG. 18 shows the formation of stabilized coacervates in extracellular Chinese hamster ovary (CHO) culture media across different PEG-diamine concentrations. Coacervate turbidity was quantified directly using diluted samples of the culture media. Below the chart are photographs of the plate wells illustrating that the level of turbidity can be seen by the naked eye.

FIG. 19 shows the difference in turbidity and the identifiable disruption of the coacervates in the oxidative environments.

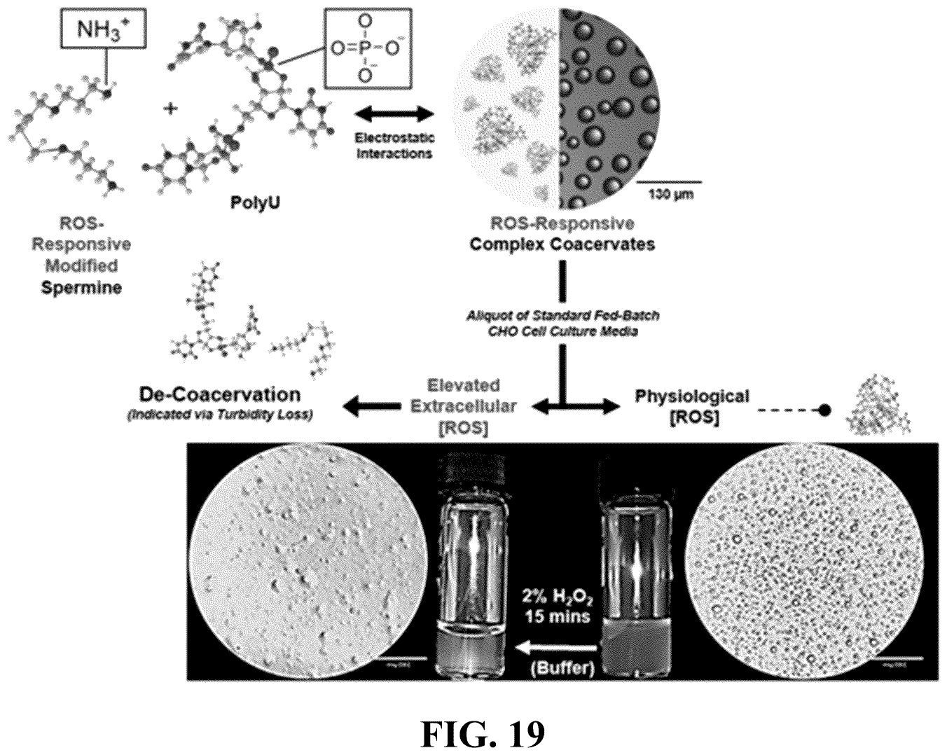

### DETAILED DESCRIPTION

The various compositions, methods and system embodiments disclosed or contemplated herein include complex coacervates compositions and methods of use. In one embodiment, the coacervates can be simple or complex coacervates. In another embodiment, the coacervate is a complex coacervate comprised of RNA and a cationic polymer. The RNA may be modified with base substitutions, modifications to the backbone, the inclusion of base analogues, or the inclusion of labels or linkers. The modifications can help prevent RNA degradation or further help direct the coacervate to the intended location within a sample or patient. The RNA can be mRNA, siRNA, miRNA, lncRNA, shRNA, crRNA, gRNA, rRNA, or tRNA

In another embodiment, cationic polymer is spermine or spermidine. In another embodiment, the cationic polymer is modified with DTSSP or dithiobis(succinimidyl propionate) (DSP/DTSP). In another embodiment, the cationic polymer is spermine modified with DTSSP at a ratio between 1:0 and 1:1 spermine/DTSSP.

In another embodiment, the coacervates are encased by a nanoparticle. The nanoparticle can have a diameter between 50 and 1000 nm. In another embodiment, the nanoparticle diameter is between 100-500 nm, or between 100-200 nm.

In another embodiment, the complex coacervate includes, or is used in tandem, with a stabilizing agent. In another embodiment, the stabilizing agent is polyethylene glycol (PEG) or PEG-diamine.

In another embodiment, compositions of the invention are used in methods for delivering RNA to a cytosol of a cell. In another embodiment, the cationic polymer is modified with DTSSP or DTSP, and the polymer is added to RNA to form a cationic polymer/RNA structure. In another embodiment, the method includes encapsulating the cationic polymer/RNA structure in a nanoparticle. The nanoparticle is contacted with a cell containing a cytosol.

In another embodiment, the cell is located in an area of heightened reactive oxygen species (ROS) levels, such as near inflammation or a disease, or a source of ionizing radiation. In another embodiment, the methods include increasing the reactive oxygen species (ROS) level of the cytosol to modulate the separation of the cationic polymer and the RNA.

In another embodiment, the coacervation and subsequent release of the RNA is monitored. In another embodiment, the monitoring is through detection of a label such as a radio label, a fluorescent label, or a sequence tag.

In another embodiment, the coacervate or nanoparticle containing the coacervate are delivered to the region of therapy via injection or intravascular administration. In another embodiment, the RNA is delivered to the cytosol for therapeutic applications. In another method, the RNA encodes for a protein, or the RNA is an inhibitor of a sequence.

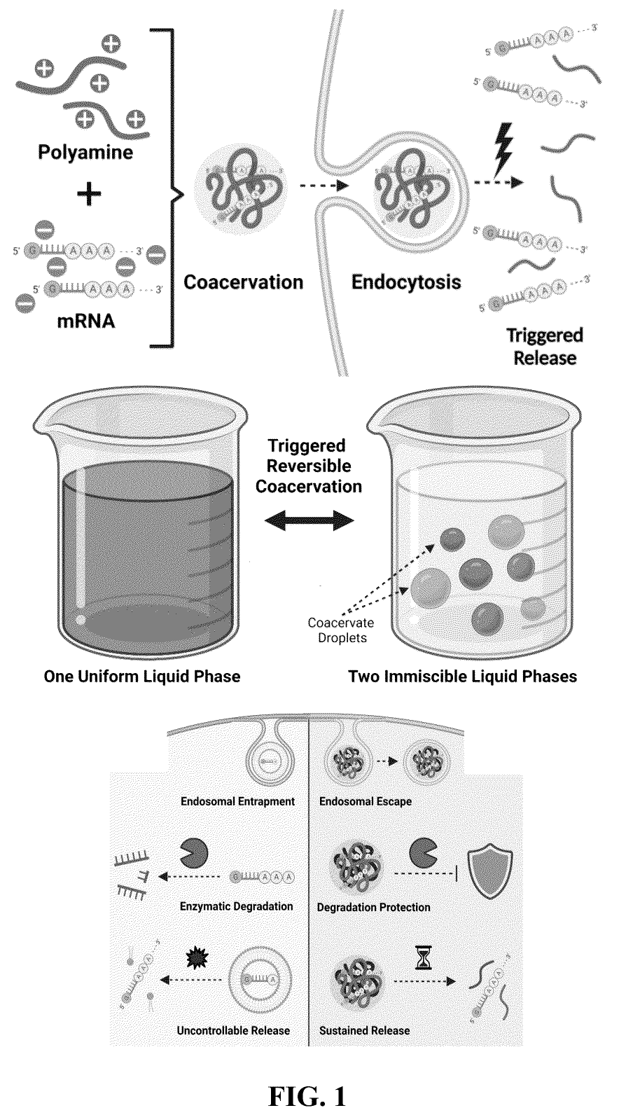

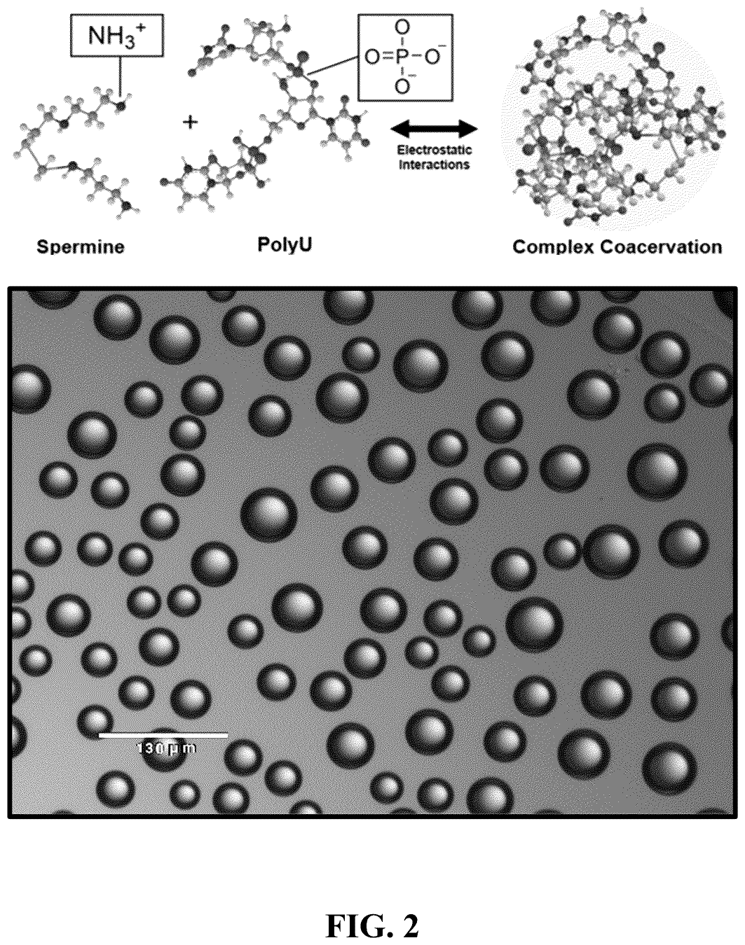

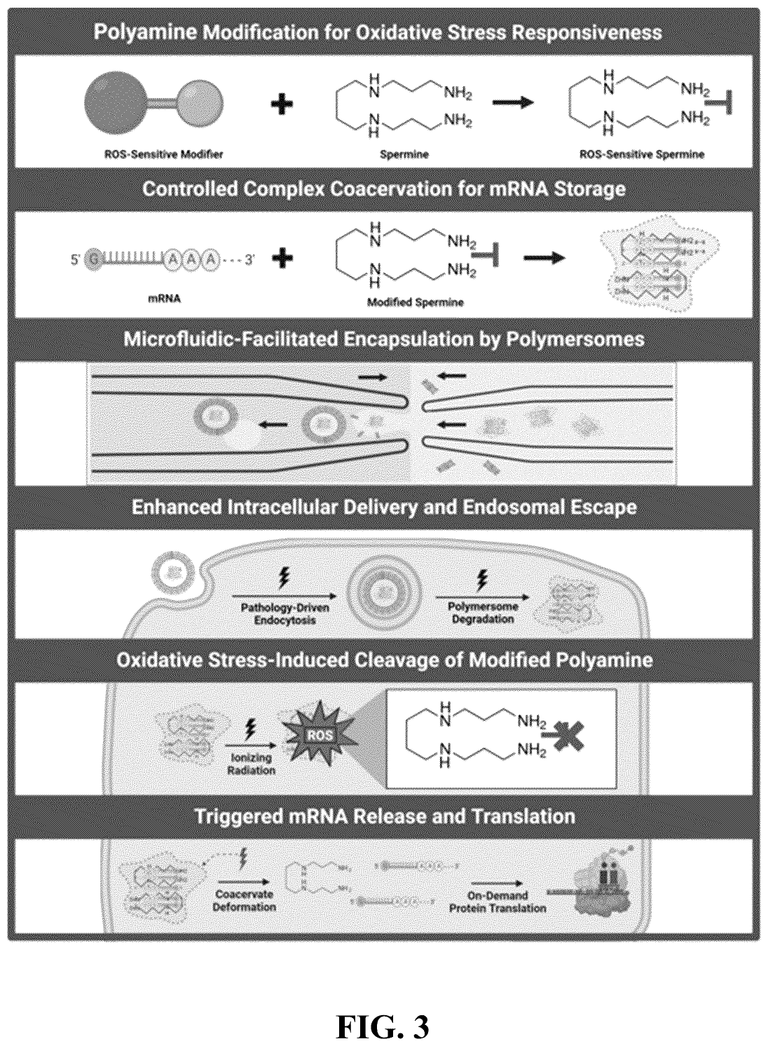

Complex coacervates are phase-separated droplets formed through the electrostatic interaction between oppositely charged polyelectrolytes (FIG. 1). In some embodiments, anionic RNA molecules are coacervated with a cationic polymer. In another embodiment, the cationic polymer is a spermine (FIG. 2) or spermidine. A crosslinking reaction is performed with the amine-reactive compound 3,3′-dithiobis(sulfosuccinimidyl propionate) (DTSSP), comprising sulfonated NHS-ester terminal groups and a central oxidizable disulfide bond. In this reaction, the primary amine groups of spermine crosslink the spermine molecules through DTSSP linkers via displacement by the activated ester groups of DTSSP. In the presence of increased oxidizing agents, the linkers on the modified spermine are disrupted by ROS forming highly reactive intermediates prone to further oxidation. As a result, the oxidation state of the coacervate structure is increased, leading to alterations in its physicochemical properties. These changes impact the electrostatic interactions between the spermine and RNA molecules, induce phase miscibility, and subsequently release RNA from the coacervate matrix (FIG. 3).

The modified coacervates remain stable in low ROS conditions but activate in the presence of elevated ROS levels, typical of oxidative stress. The present disclosure details precision delivery systems to ensure RNA molecules are accurately released in cells facing oxidative stress, e.g., in cancer, neurodegenerative diseases, vaccine- or inflammation-triggered ROS, and heart conditions. This selective approach enhances gene therapy efficacy in target cells, minimizing off-target effects elsewhere. Targeted delivery of RNA molecules to cells undergoing oxidative stress is achieved through strategies like redox-responsive nanoparticles, monoclonal antibodies that target oxidative stress markers, and pH-responsive hydrogel-based delivery systems that respond to acidic tumor microenvironments.

In one embodiment, a composition for RNA therapy is provided, the composition comprising an RNA and spermine to form an RNA/spermine complex coacervate. In another embodiment, the spermine is modified with 3,3′-dithiobis(sulfosuccinimidylpropionate) (DTSSP). In another embodiment, the spermine/DTSSP molar ratio is between 1:0 and 1:1. In another embodiment, the RNA molecules may be further modified to prevent degradation, such as, for example, with 2′-O-methyl groups or phosphorothioate bonds. The RNA molecules can also be labeled with a moiety that can be traced, such as, for example, with a fluorophore, a radiolabel, or a sequence tag.

In another embodiment, a method of RNA-based therapy is provided, the method comprising providing an RNA/spermine structure in a nanoparticle to a cytosol of a cell and increasing ROS levels to release the RNA molecules. In another embodiment the ROS levels are provided by ionizing radiation to the cell. In another embodiment, the spermine is modified with DTSSP, and in another embodiment the nanoparticle encapsulation is facilitated using microfluidics. ROS levels can also be increased as the coacervate comes into proximity to a vaccine, disease or inflammation.

In another embodiment, methods are provided for non-destructive sensing of ROS in cell cultures to detect oxidative stress states in real-time. In certain embodiments, the cell cultures are Chinese Hamster Ovary (CHO) or other mammalian cell cultures. ROS can be detected visually and responses can be automated into biomanufacturing workflows to monitor and adjust process parameters such as media composition, antioxidant supplementation, or stressor exposure. The monitoring methods can provide feedback-controlled release systems for protective or regulatory RNAs (e.g., miRNAs) in cell culture environments experiencing oxidative stress. In certain embodiments, labeled RNAs (e.g., polyU-AlexaFluor568) can be used to visualize RNA recruitment and release, providing a fluorescence-based readout of ROS levels in situ.

The following examples will detail the modification of a cationic polymer such as spermine, the coacervate phase separation and coacervated RNA's response to oxidative stress.

### Example 1

The following example describes the modification of spermine and its influence on coacervate capability.

Polyuridylic acid (polyU) potassium salt (MW 600-1000 kDa), spermine, 3,3′-dithiobis(sulfosuccinimidylpropionate) (DTSSP), formic acid (CH₂O₂), hydrogen peroxide solution (H₂O₂) (30%), magnesium chloride (MgCl₂), methanol (CH₃OH), polyethylene glycol (PEG) (MW 6000 Da), and 4-(2-hydroxyethyl)-1-piperazineethanesulfonic acid (HEPES) were purchased from Sigma Aldrich (St. Louis, MO). PEG-diamine (MW 6000 Da) was purchased from Polysciences (Warrington, PA). Deionized (DI) water was obtained for aqueous solutions using a Millipore water filtration system. Deuterium oxide was purchased from Acros Organics (Geel, Belgium).

Spermine was reacted with DTSSP in a 5 mM HEPES and 1 mM MgCl₂ solution (pH=7.4) at varying molar ratios (spermine:DTSSP), including: 1:0 (unmodified), 1:0.125, 1:0.25, and 1:0.5. The solutions were gently mixed via pipetting and allowed to react at room temperature in the dark for 30 minutes.

Tunable Extent of Spermine Modification via Mass Spectrometry. Pure spermine and DTSSP, along with modified spermine composed of crosslinking molar ratios of 1:0.125, 1:0.25, and 1:0.5 (synthesized by above methods) were diluted 100× in 10% aqueous methanol from 7 mg/mL standard solutions. Diluted samples were injected (10 μL) with an Agilent 1290 Infinity II UHPLC equipped with Agilent ZORBAX Extend-C18 column (2.1×50 mm, 1.8 μm) maintained at 40.0° C. The 10-minute gradient is displayed in Table 1; Mobile Phase A was water (0.1% formic acid) and Mobile Phase B was methanol. Following chromatographic separations, samples were ionized with an Agilent Jetstream Source (AJS) operated in positive electrospray mode and then analyzed with an Agilent 6560 IM-QTOF operated in QTOF-only mode. High-resolution mass spectra were acquired with mass accuracy <5 ppm and all data was interrogated using Agilent MassHunter Qualitative Analysis software. Relative abundance was measured by integration of peak area in extracted ion chromatograms.

TABLE 1
LC Gradient Conditions

| Time (min) | MP A % | MP B % |
|---|---|---|
| 0.00 | 90 | 10 |
| 2.00 | 90 | 10 |
| 3.00 | 40 | 60 |
| 8.00 | 0 | 100 |
| 9.00 | 0 | 100 |
| 9.01 | 90 | 10 |

Characterization of Modified Spermine via NMR. NMR experiments were conducted on a Bruker AVANCE NEO (NanoBay) 300 MHz spectrometer (Bruker BioSpin, Billerica, MA) equipped with a 2-channel UltraShield multinuclear Broad Band (109Ag-31P and 19F) probe (BBFO). Spermine modified with a 1:0.125 (spermine:DTSSP) ratio was lyophilized and then resuspended in deuterium oxide at a concentration of 10 mg/mL. Pure, unmodified spermine and DTSSP were also separately suspended in deuterium oxide at the same concentration. Proton (\hat{ }1H) NMR spectra were acquired at 298 K using standard pulse sequences provided by the manufacturer. The spectral width was set to 15 ppm, and 16 scans were accumulated to ensure an adequate signal-to-noise ratio. Data was processed using Bruker TopSpin 4.0 software (Bruker BioSpin, Billerica, MA), and spectra were referenced to the residual solvent signal of deuterium oxide.

Modification Influence on Coacervate Capability. Final concentrations of polyU in solution for each sample were 0.05 wt %. Final concentrations of unmodified and modified spermine in polyU coacervates ranged from 0.005 wt % to 0.04 wt %. Coacervates were prepared in a 5 mM HEPES (pH 7.4), 1 mM MgCl2 buffer¹⁰. Stock solutions were added in the following order for preparation of each sample: deionized water, HEPES, MgCl2, polyU, spermine, and mixed via gentle pipetting in between addition of each stock solution. Samples were incubated at 37° C. for five minutes with moderate shaking. The amount of unmodified and modified spermine required for coacervation was determined using UV-visible spectroscopy via the BioTek Synergy H1M plate reader by measuring the sample absorbance/transmittance at 500 nm and converting to turbidity. Curve fitting of turbidity plots were performed using the four-parameter logistic nonlinear regression model (also known as the Hill equation or the variable slope sigmoidal equation) (eqn (1))¹⁰ in Python 3.8. Phase contrast images were taken with an Echo Revolve hybrid microscope.

f(x)=base+((max−base)/(1+[xhalf/x]\hat{ }rate)) (1)

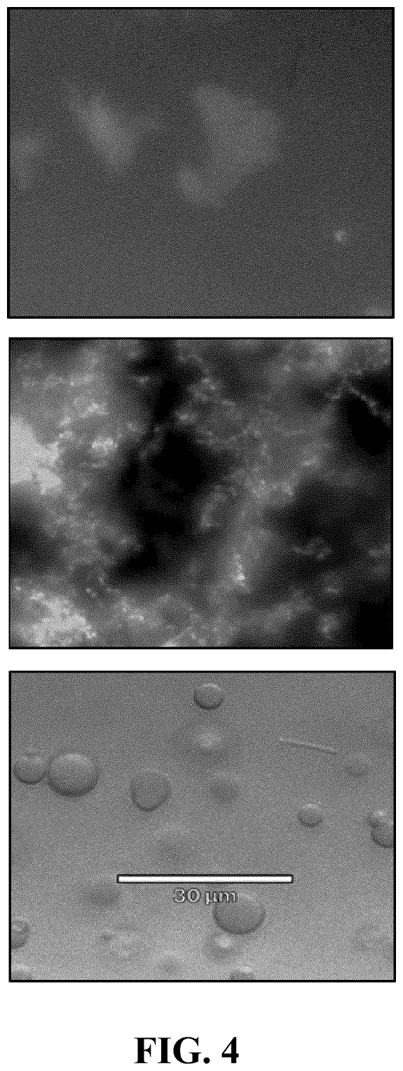

To confirm the coacervation ability of the modified spermine with larger and more complex RNA molecules, preliminary experiments were conducted with Cyanine 5 (Cy5)—Tagged Green Fluorescent Protein (GFP)—Encoding mRNA and increasing concentrations of spermine modified with a spermine:DTSSP molar ratio of 1:0.25. The results were visualized using phase contrast imaging, with spermine concentration increasing from top to bottom image (FIG. 4).

### Example 2

The following example describes the impact of polyethylene glycol derivatives on coacervate stability, and the redox-responsivity of modified and stabilized coacervates.

To evaluate the stabilizing effect of polyethylene glycol derivatives, coacervates were prepared as described previously using unmodified or modified spermine and polyU at final concentrations of 0.02 wt % and 0.05 wt %, respectively, in 5 mM HEPES (pH 7.4), 1 mM MgCl₂ buffer. Stabilizing agents were added at 0.1 wt % final concentration either as PEG-diamine (6 kDa) or PEG (6 kDa) after the addition of polyU but prior to spermine addition. The coacervates were incubated at 37° C. for five minutes with moderate shaking. Turbidity measurements were collected over time using a kinetic measurement (at 37° C. and with moderate shaking) on the BioTek Synergy H1M plate reader at 500 nm to monitor coacervate disassembly. Rate of turbidity change was calculated from the linear region of each curve and normalized to the rate of unmodified coacervates under the same stabilization environmental conditions. Phase contrast microscopy images were taken using an Echo Revolve hybrid microscope.

Redox-Responsivity of Modified Coacervates. Modified spermine was reacted at different molar equivalents (1:0.125, 1:0.25, and 1:0.50, spermine:DTSSP) prior to coacervate formation as described previously. Final concentrations of polyU and modified spermine were 0.05 wt % and 0.02 wt %, respectively. Coacervates were prepared in 5 mM HEPES (pH 7.4), 1 mM MgCl₂ buffer using the same addition sequence and mixing as described previously. Samples were incubated at 37° C. for five minutes with moderate shaking, followed by treatment with either 0.5 M, 1.0 M, or 1.67 M final concentration hydrogen peroxide to initiate a oxidizing environment. Turbidity changes over time were measured as described previously using the BioTek Synergy H1M plate reader, and rates of turbidity loss were normalized to unmodified spermine coacervates. Phase contrast images were captured with an Echo Revolve hybrid microscope immediately after the kinetic measurements finished.

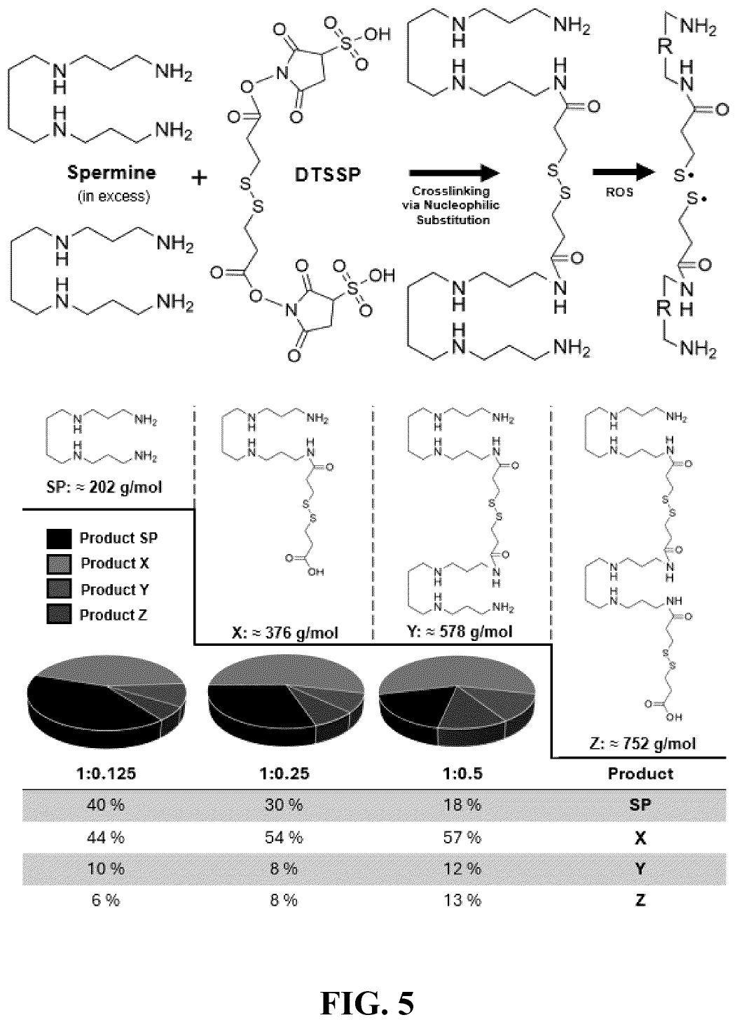

Redox-Responsivity of Stabilized Coacervates. To test how PEG and PEG-diamine influence the redox-responsivity of modified coacervates, coacervates were formed with 0.05 wt % polyU and 0.02 wt % unmodified or modified spermine at three modification extents (1:1.125, 1:1.25, and 1:1.50). PEG (6 kDa) or PEG-diamine (6 kDa) was added at 0.1 wt % final concentration during the preparation process, immediately after polyU addition. Coacervates were incubated at 37° C. with moderate shaking for five minutes before treatment with 1.67 M hydrogen peroxide. Turbidity measurements at 500 nm were collected over time using a BioTek Synergy H1 M plate reader. The rate of turbidity change was determined and normalized to the unmodified spermine coacervates for each stabilizer condition. Phase contrast images of the coacervates following oxidation were taken using an Echo Revolve hybrid microscope. Full image sets of all modification levels and stabilizer conditions are provided in Supplemental FIG. 5.

Statistical Analyses. Statistical analyses were performed for each experiment in accordance with the most appropriate statistical test. Normalized turbidity changes and rates of change were analyzed using a two-way ANOVA with Tukey test to correct for multiple comparisons with a threshold p-value of 0.05. All statistical analyses were performed using GraphPad Prism 10.

Spermine Modification for Redox-Responsivity: To instill redox-responsiveness in the coacervate system, spermine was reacted with the crosslinking agent DTSSP, comprising an amine-reactive N-hydroxysulfosuccinimide (sulfo-NHS) ester at each end of a spacer arm containing a central oxidizable disulfide bond. During this reaction, the primary amine groups of spermine act as nucleophiles, crosslinking the spermine through the DTSSP linker by attacking the activated ester groups of DTSSP and displacing the sulfo-NHS groups (FIG. 5). The oxidizing environment induced by excess ROS may impact the stability of coacervates formed with the modified spermine. The presence of increased oxidizing agents promotes the disruption of disulfide bonds, potentially resulting in conformational changes that weaken the interactions between spermine and polyU, causing the polyU molecules to be released from the coacervate matrix. To optimize coacervation, the degree of crosslinking is delicately balanced to maintain a residual charge that will govern RNA-spermine interactions (FIG. 2). While the crosslinking reaction may result in an overall reduced charge on the modified spermine, both residual charge and other intermolecular interactions (such as hydrophobic interactions and hydrogen bonding) can still facilitate the coacervation process, taking into account potential intra- and intermolecular crosslinking reactions. To address the balance between degree of crosslinking and residual charge, spermine was reacted with various initial DTSSP concentrations including 1:0.125, 1:0.25, and 1:0.5 (spermine:DTSSP molar ratios), to vary the likelihood of intermolecular crosslinking and the availability of primary amines for coacervation interactions.

Tunability of Spermine Modification Extent: To verify the successful crosslinking of spermine with DTSSP and to determine the extent of modification, liquid chromatography coupled to high-resolution mass spectrometry (LC-HRMS) was utilized to analyze reaction products with varying reactant spermine:DTSSP molar ratios (1:0.125, 1:0.25, and 1:0.5). This method provided insight into the modification products, identified by their distinct molecular weights, and corroborated the proposed mechanism of DTSSP-mediated crosslinking (FIG. 5). The [M+H]⁺ ion of unmodified spermine was observed at m/z 203.223, with its abundance decreasing consistently as the concentration of DTSSP increased, suggesting effective crosslinking. The products of the reaction were characterized by their expected molecular weights. Product X (Spermine+DTSSP Crosslink) at m/z 377.205, corresponds to the initial crosslinking of a single spermine molecule with DTSSP. This product's abundance is increased as the concentration of DTSSP is increased, indicating a proportional relationship between DTSSP concentration and the formation of crosslinked spermine. Product Y (Spermine x2+DTSSP Crosslink) at m/z 579.419, represents the formation of a structure between two spermine molecules and one DTSSP linker. The quantity of this product showed a modest increase with increasing DTSSP concentration, reflecting the higher probability of intermolecular crosslinking as the DTSSP concentration increases. Product Z (Spermine x2+DTSSP Crosslink x2) at m/z 753.399, is indicative of extensive crosslinking involving two spermine molecules and two DTSSP linkers. The abundance of this product also increased with DTSSP concentration. These MS results demonstrate the effectiveness of crosslinking mechanism, showing that the reaction favors the formation of larger and more complex crosslinked structures, particularly at higher DTSSP concentrations. The oxidizable disulfide bond within DTSSP is effectively utilized in modifying spermine, which is essential for subsequent oxidative stress-responsive behaviors.

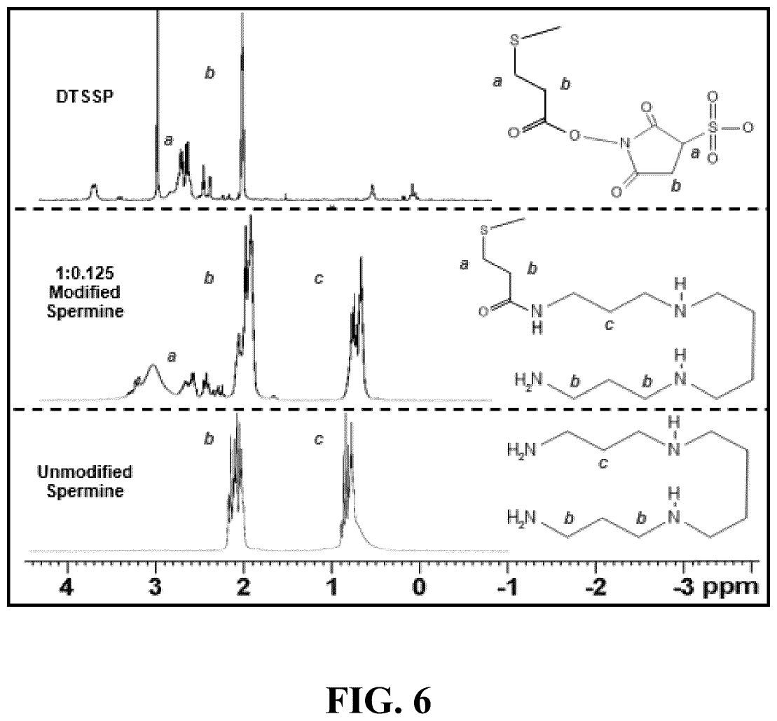

Similar and small molecular sizes of the reactants and products underwent additional analysis. Proton nuclear magnetic resonance (\hat{ }1H NMR) spectroscopy was also explored to confirm the chemical modifications, though signal overlap and sample complexity limited the interpretability of the spectra (FIG. 6). The spectra contained signals from both unmodified and modified spermine, as well as residual DTSSP and its byproducts, and the observed trends are broadly consistent with the proposed crosslinking mechanism and support the hypothesis of successful spermine modification. Additionally, these results support the hypothesis that the modification process does not compromise the structural integrity required for the spermine's interaction with RNA within the coacervate system.

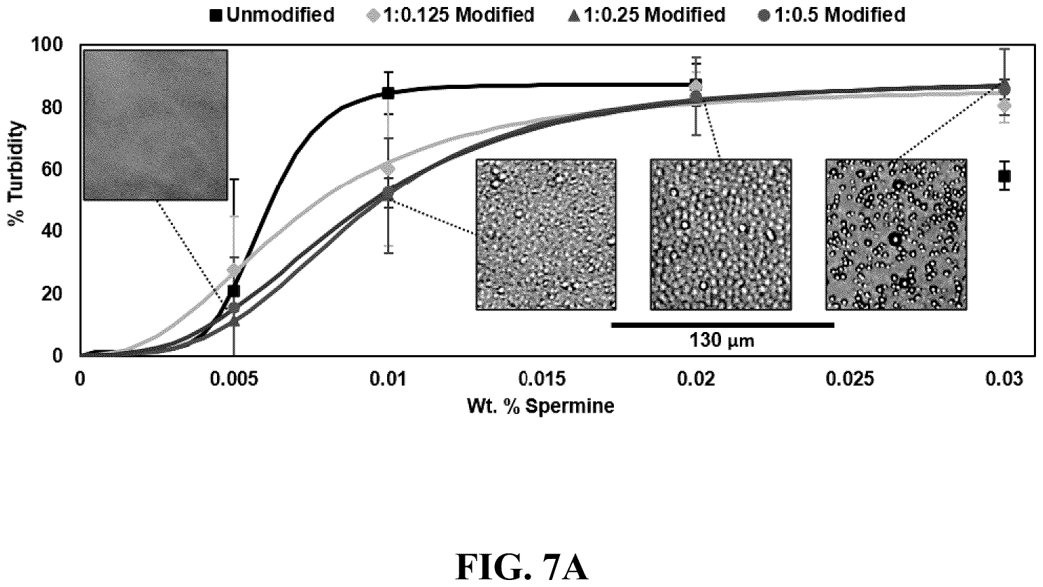

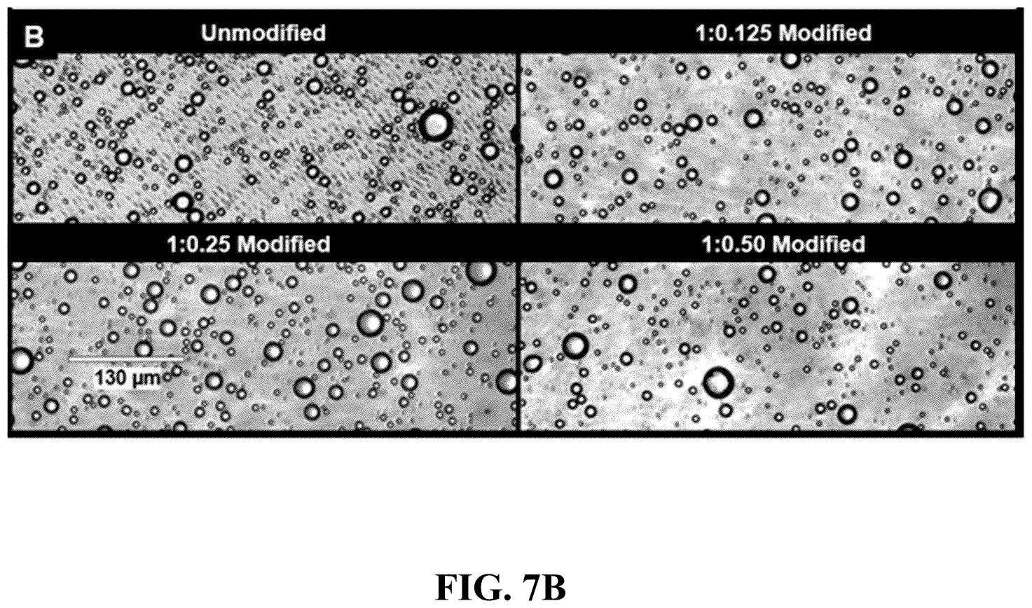

Modification Influence on Coacervation Capability: After confirming the successful modification of spermine, the extent of this modification's effect on spermine's capacity to form coacervates with polyU was examined. The baseline was established for the modified samples by determining the minimum amount of unmodified spermine needed to achieve coacervation with a fixed amount of polyU. This was quantified through turbidity measurements to assess phase separation (FIG. 7A). Unmodified spermine (squares) coacervated with polyU (turbidity≈80%) at a minimum concentration of approximately 0.01 wt % spermine, consistent with prior literature findings. Since the modified spermine is expected to have a slightly reduced charge compared to its unmodified counterpart, a range of final modified spermine concentrations were explored. spanning 0.005 wt % to 0.03 wt %. The results indicated that the modified spermine (with diamonds representing 1:0.125, triangles as 1:0.25, and circles as 1:0.5) effectively coacervated with polyU (turbidity≈80%) at a minimum concentration of approximately 0.02 wt % for all tested modification ratios, suggesting that the modification has only a marginal impact on the overall charge of spermine and its capacity to form coacervates with polyU.

To further characterize the effect of this modification on coacervate formation, we evaluated the size and polydispersity of coacervates formed by unmodified and highly modified spermine using dynamic light scattering (DLS) (FIG. 7B). This analysis provides insight into whether the chemical modification alters the physical properties of the resulting coacervates. The z-average hydrodynamic diameter of coacervates formed with unmodified spermine was 1.623±0.082 μm, while coacervates formed using the highest extent of modification (1:0.50) exhibited a comparable size of 1.557±0.202 μm. Similarly, the polydispersity index (PDI) showed no significant change upon modification, decreasing slightly from 0.319±0.254 in the unmodified sample to 0.232±0.136 in the modified condition. These results demonstrate that the introduction of the redox-responsive modification does not significantly impact the size or uniformity of the coacervate phase, indicating that the phase behavior and molecular organization of the system are largely preserved. This supports the conclusion that the chemical modification strategy retains the essential physicochemical features required for coacervation, while imparting additional functionality through redox-responsivity.

Impact of Polyethylene Glycol Derivatives on Coacervate Stability: While the chemical modification did not significantly alter the size or uniformity of the resulting coacervates, long-term stability remains a key consideration for practical applications. To evaluate whether the redox-responsive modification affects coacervate longevity, and to explore potential strategies for enhancing stability, the impact of adding low concentrations (0.1 wt %) of polyethylene glycol (PEG) and PEG-diamine (PEG-DA) was investigated, both with a molecular weight of 6 kDa, as free stabilizing agents. Free PEG (6 kDa) is a neutral, hydrophilic polymer that primarily exerts a steric stabilization effect, potentially creating a hydration shell around the coacervate droplets that limits fusion and sedimentation without engaging in strong intermolecular interactions. Both PEG and PEG-DA are expected to contribute steric stabilization through their polymeric backbones, however PEG-DA also features terminal amine groups that may enable additional electrostatic interactions with charged components. By comparing these two additives, it could be determined if differences in interaction potential—steric exclusion alone versus steric exclusion plus potential electrostatic interactions—could influence coacervate stability, particularly in systems incorporating redox-responsive modifications that may alter droplet cohesion over time.

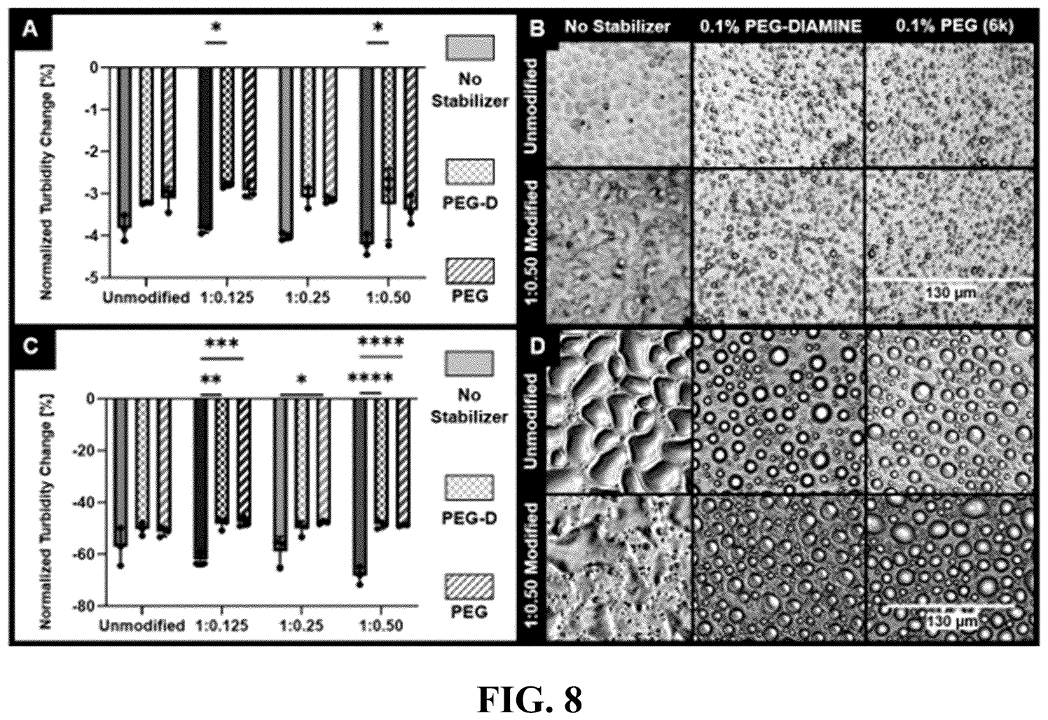

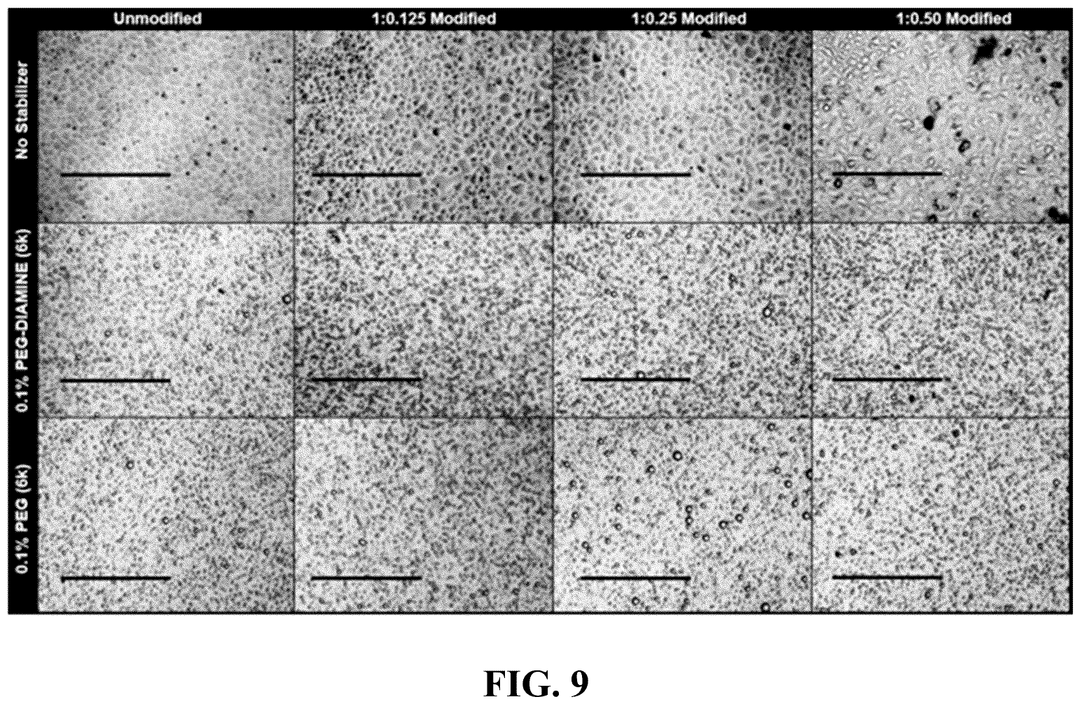

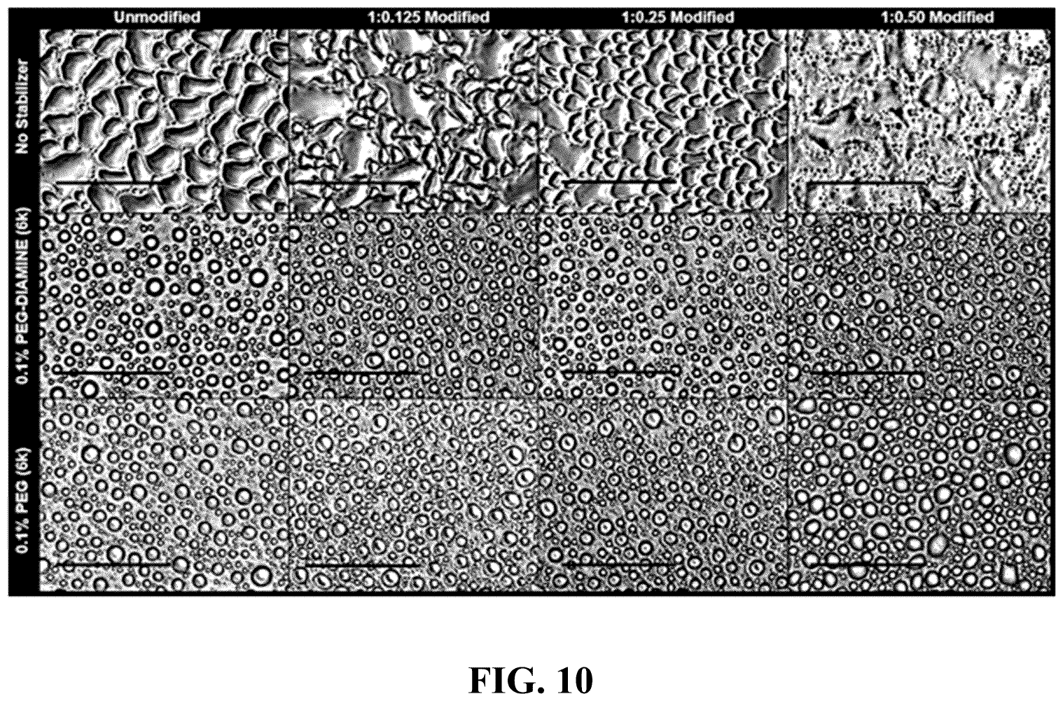

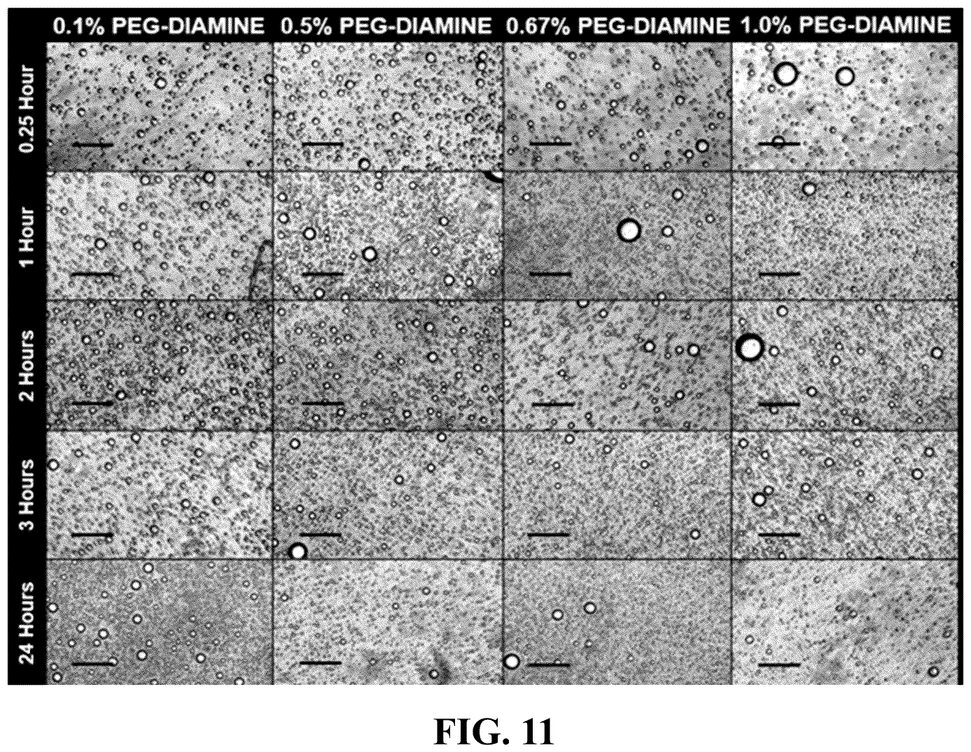

To directly evaluate the impact of PEG and PEG-DA on coacervate retention and morphology, the normalized change in turbidity over time was assessed as a proxy for coacervate stabili^{t}y, alongside phase contrast imaging to visualize droplet persistence and morphology (FIG. 8). Turbidity measurements at 15 minutes and 24 hours (FIG. 8, Boxes A and C) demonstrate that both PEG and PEG-DA significantly reduce coacervate degradation compared to unstabilized samples to a similar extent, regardless of the extent of spermine modification. The consistency of this trend over time suggests that the stabilizers exert a sustained effect, beginning at the point of coacervate formation and continuing through extended incubation. These observations are further supported by phase contrast microscopy. After just 15 minutes (FIG. 4, Box B), both unmodified and modified coacervates lacking a stabilizer show evidence of droplet fusion, flattening, or incomplete coacervate formation. In contrast, the addition of either stabilizer promotes the formation of well-defined, spherical droplets. This stabilization persists at 24 hours (FIG. 4, Box D), where coacervates in all stabilized conditions retain their morphology and droplet density, despite slight increases in droplet size and signs of minor coalescence. These results collectively indicate that both PEG and PEG-DA effectively preserve coacervate integrity over time, beginning immediately after formation and extending through at least 24 hours. Notably, this effect is consistent across all levels of spermine modification, indicating that the redox-responsive chemical changes do not impair the ability of either excipient to stabilize the coacervate phase (FIG. 9-10). Furthermore, FIG. 11 shows that increasing the concentration of PEG-diamine does not alter this trend, indicating that the stabilizing effect is maintained across a range of concentrations. These results suggest that our modified coacervates remain compatible with standard formulation additives and underscore the modularity of this platform for applications requiring enhanced structural longevity.

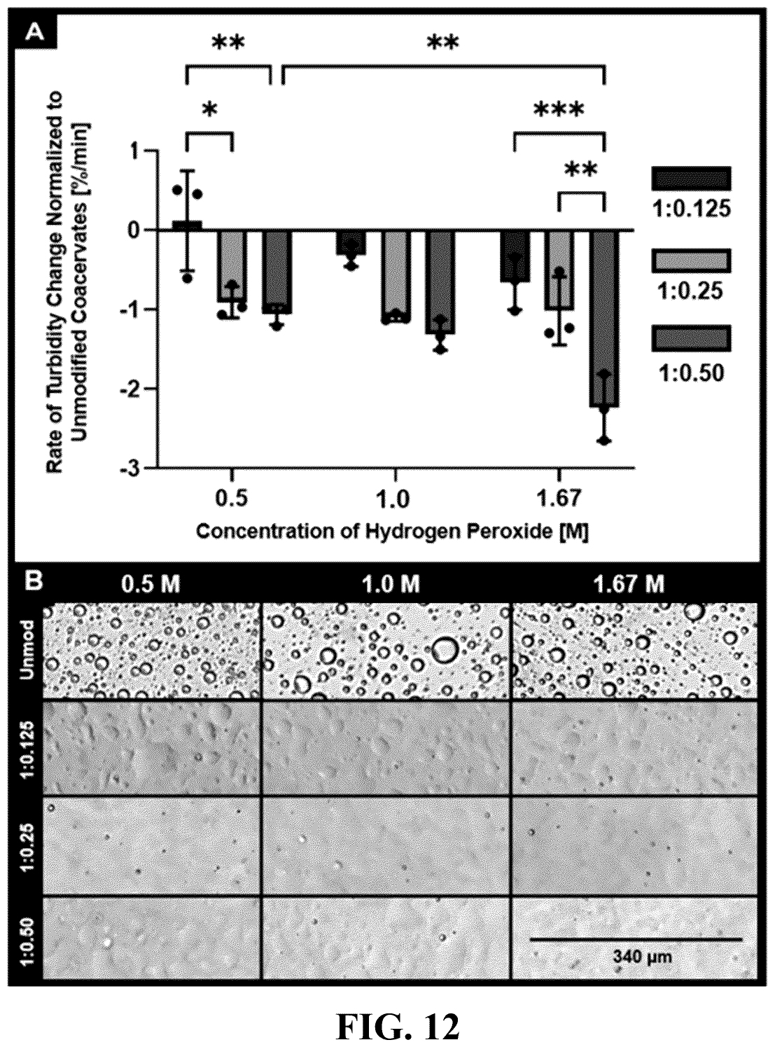

Redox-Responsivity of Modified Coacervates: To determine whether the DTSSP modification instills redox-responsivity into the coacervate system, we exposed coacervates formed from either unmodified or modified spermine to varying concentrations of hydrogen peroxide (H₂O₂), a model reactive oxygen species (ROS). This experiment was designed to assess the extent to which the disulfide disruption within the crosslinking agent impacts the integrity of the coacervate phase. Coacervates were first formed under standard conditions and subsequently exposed to a range of H₂O₂ concentrations. Turbidity was monitored over a 10-minute period to quantify the rate of coacervate destabilization as a function of oxidative stress. As shown in FIG. 12, Box A, the turbidity loss rate (normalized to the unmodified coacervates) increases with both the extent of spermine modification and the concentration of hydrogen peroxide. The coacervates modified with 1:0.125 spermine:DTSSP ratio show moderate sensitivity to H₂O₂, while those modified at 1:0.25 and 1:0.5 demonstrate increasingly accelerated turbidity decay rates, indicating faster droplet disassembly. This suggests that higher degrees of crosslinking introduce more disulfide linkages susceptible to ROS-mediated disruption, thereby rendering the coacervates more response to redox conditions. Complementary phase contrast microscopy (FIG. 12, Box B) visually supports this trend. After 10 minutes of H₂O₂ exposure, unmodified coacervates retain their morphology and remain largely intact, whereas modified coacervates show diminished droplet number and definition at even the smallest modification extent. These results confirm that the DTSSP-based modification introduces a tunable redox-responsiveness into the spermine/polyU coacervate system. This behavior is consistent with the disruption of disulfide bonds under oxidative conditions, which disrupts intermolecular interactions necessary for phase separation. Importantly, the observation that unmodified coacervates remain unaffected under identical conditions underscores the specificity of this redox-triggered effect to the chemical modification strategy. This redox-sensitive behavior positions the system as a promising candidate for applications in which selective, stimulus-responsive release is desired.

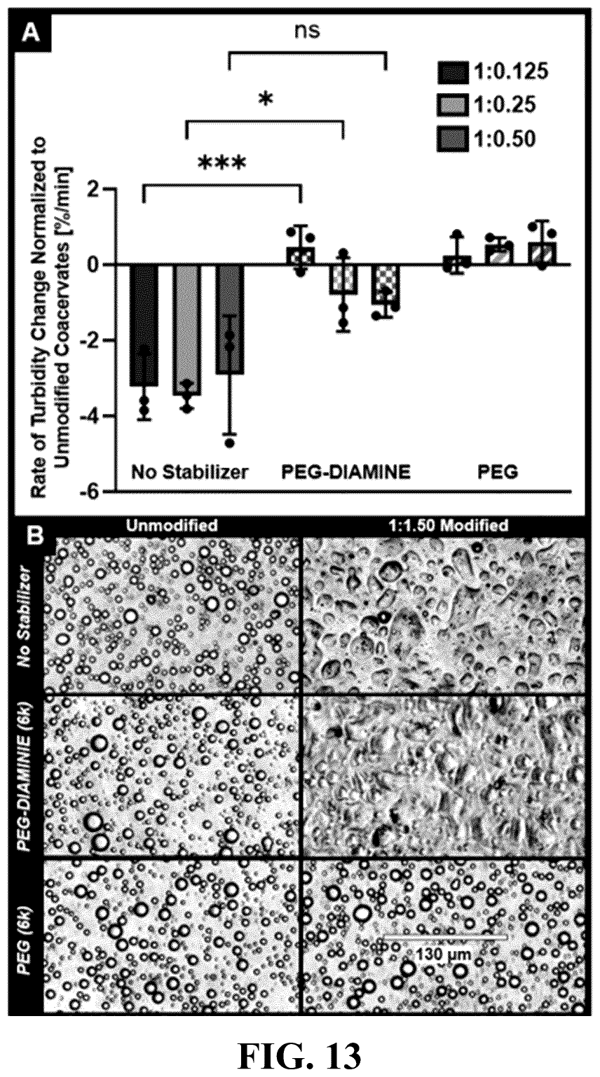

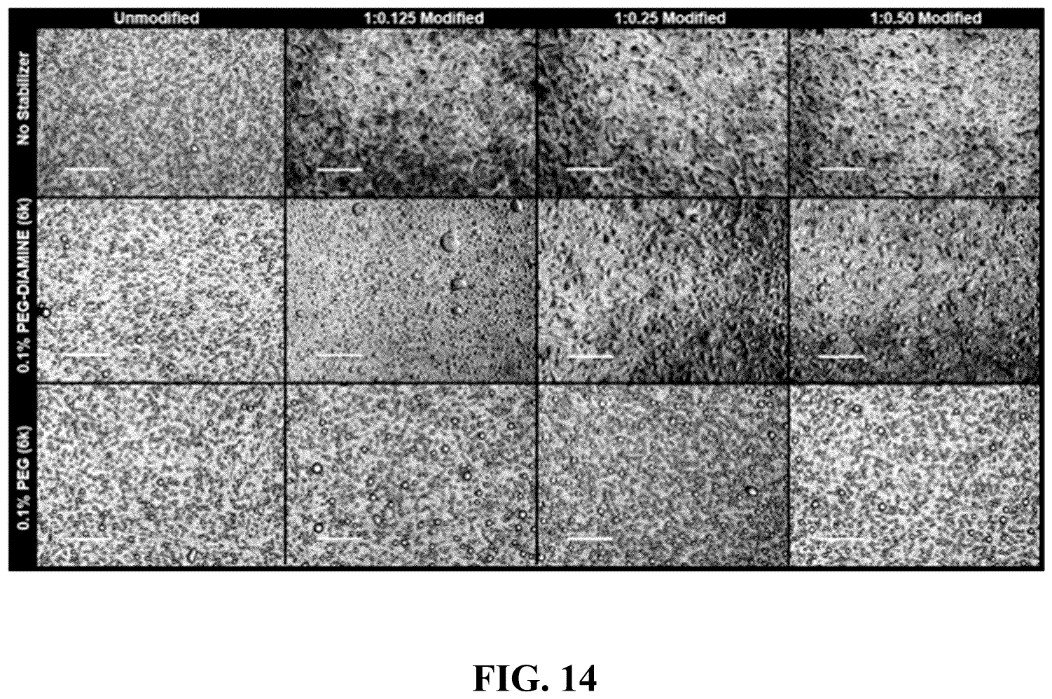

Redox-Responsivity of Stabilized Coacervates: To evaluate how coacervate stabilization strategies affect redox-responsivity, the turbidity decay assay was repeated in the presence of 1.67 M hydrogen peroxide—the highest concentration tested—using either no stabilizer, 0.1 wt % PEG, or 0.1 wt % PEG-DA. The rate of turbidity change was normalized to unmodified coacervates at each crosslinking extent (FIG. 13, Box A). In line with previous results, coacervates with no stabilizer showed strong redox-responsivity across all modification levels. PEG-DA stabilized coacervates retained partial responsivity, although to a significantly lesser extent, while PEG-stabilized coacervates showed almost complete inhibition of turbidity decay. This trend suggests that PEG alone introduces steric hindrance that effectively blocks destabilization, while PEG-DA despite similar steric bulk, preserves some responsiveness—likely due to its terminal amines enabling electrostatic interactions with the coacervate matrix. Phase contrast microscopy (FIG. 13, Box B) reinforces this interpretation. Modified coacervates with no stabilizer or PEG-DA visually disassemble after H₂O₂ exposure, while PEG-stabilized samples maintain their droplet morphology. Interestingly, at the lowest modification extent (1:0.125), PEG-DA did not produce a strong turbidity response, though supplemental microscopy (FIG. 14) shows visible structural disruption, implying that responsivity still occurs but may fall below the threshold of the bulk turbidity assay. These findings support the idea that while steric shielding from PEG can block redox-triggered destabilization, PEG-DA's potential for additional electrostatic interactions helps preserve responsiveness—especially at higher crosslinking densities where the destabilization mechanism is stronger.

This presents a strategy for engineering redox-responsivity into polyU-based coacervates by chemically modifying spermine with the disulfide crosslinker DTSSP. Through systematic variation in reaction stoichiometry. Spermine crosslinking can be precisely tuned to introduce disulfide linkages while preserving the phase separation capability with polyU. Mass spectrometry confirmed the formation of crosslinked products, with higher DTSSP concentrations yielding more complex conjugates, supporting the redox-sensitive modification mechanism. Despite a partial reduction in charge density, modified spermine maintained its ability to coacervate with polyU, with no significant impact on coacervate size or uniformity, demonstrating the system's robustness. Stabilization with PEG and PEG-diamine extended coacervate persistence, with PEG-diamine preserving redox-responsivity due to its electrostatic interactions, unlike PEG, which blocked the redox response. This highlights the importance of excipient selection for responsive systems. The introduction of disulfide bonds enabled concentration-dependent destabilization upon hydrogen peroxide exposure, confirming controlled release under oxidative stress.

Overall, the invention provides a straightforward, tunable approach for engineering redox-responsive coacervates, addressing key challenges associated with traditional systems, such as the reliance on synthetic carriers that require full degradation and the tradeoff between stability and responsiveness. By using spermine, a biocompatible molecule naturally found in the body, compatibility with biologically relevant polyanions like RNA is maintained while introducing oxidative stress sensitivity. The ability to fine-tune the modification extent ensures precise control over stability and release profiles, making this system adaptable to various functional needs. Furthermore, the system's compatibility with standard stabilizers like PEG and PEG-diamine underscores its versatility, enabling easy adaptation for different formulations. The compositions and methods of the invention not only advance the understanding of coacervate chemistry but also provide a foundation for developing smart, biocompatible materials with dynamic, stress-responsive behavior. With its tunability, stability, and potential for optimization, this system is well-suited for applications in biomanufacturing, drug delivery, and other areas requiring responsive and biocompatible materials.

### Example 3

The following example details the response of the coacervated RNA to radiation. A custom-built X-ray cabinet designed to emit low-dose Bremsstrahlung X-rays, which, upon interaction with fluorescent copper plates, generate quasi-monochromatic characteristic radiation (Sengupta, B., Medlin, D., Sprunk, M., Napolitano, J., D'Avanzo, J., Ran Zheng, X., Dean, D. & Takacs, E. X-ray cabinet to deliver highly characterized low-dose soft x-ray radiation to biological samples. Rev. Sci. Instrum. 91, (2020)).

X-Ray Cabinet Irradiation: Irradiation was conducted within a controlled climate using a radiation-shielded incubator cabinet that emits low-dose Bremsstrahlung x-rays, which, upon interacting with interchangeable fluorescent copper plates, generate robust and quasi-monochromatic characteristic radiation. Both modified and unmodified coacervates containing spermine at a crosslinking extent of 1:0.25 were irradiated at a dose rate of 0.1422 mGy/min for either 5 or 10 minutes resulting in total absorbed doses of 0.711 mGy and 1.422 mGy

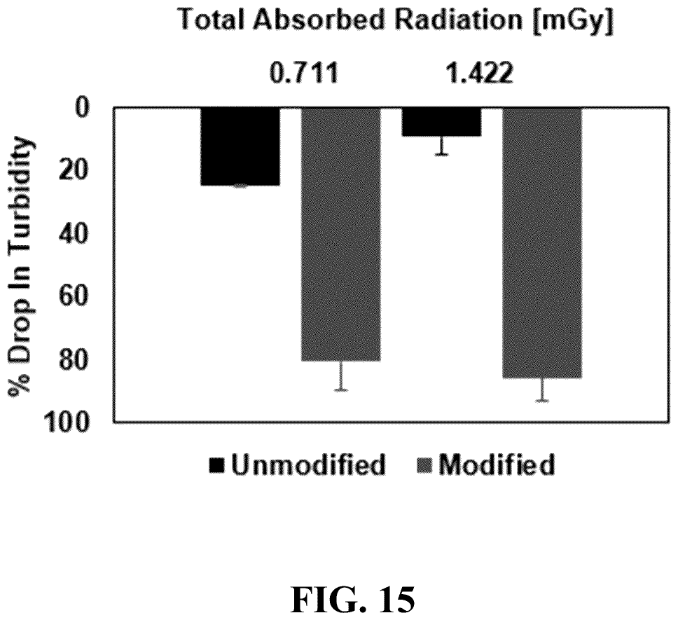

The experimental setup allowed for the experiment to induce ROS production and illuminate oxidative stress environments for investigation. To assess the stability of the modified coacervates in the presence of ROS, coacervates composed of both modified and unmodified spermine were subjected to irradiation for five and ten minutes at a rate of 0.142 mGy/min. This resulted in absorbed doses of 0.711 mGy and 1.422 mGy, respectively. The changes in turbidity were measured to gain insights into their response to ionizing radiation (FIG. 15). After irradiation with 0.711 mGy, a significant reduction in turbidity for the modified coacervates was observed, with a decrease of approximately 80.55% (+/−9.29%) shown in red. In contrast, the unmodified samples only exhibited a modest decrease of about 24.64% (+/−0.24%), represented in black. A similar trend was observed in samples irradiated with 1.422 mGy, where the modified coacervates displayed a substantial drop in turbidity of approximately 85.57% (+/−7.30%) in red, while the unmodified ones showed a much smaller decrease of 9.38% (+/−5.78%) in black. These pronounced reductions in turbidity following irradiation suggest that the modification induces a responsive structural change in the coacervates, ultimately leading to phase miscibility. In an oxidative-stressed cellular environment, these structural alterations and resulting coacervate miscibility should lead to the release of RNA molecules from the phase-separated structure in response to oxidative stress.

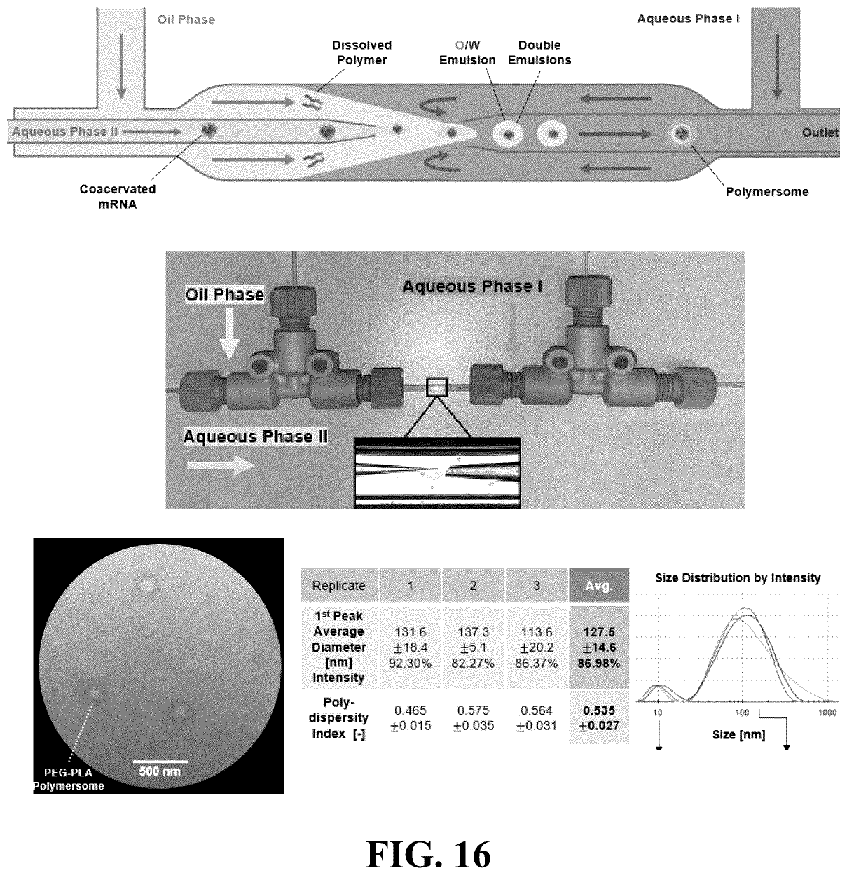

Additional work was conducted to explore the potential of the modified coacervates for co-delivery via nanoparticles, aiming to enhance intracellular delivery and maintain phase separation upon delivery for more effective therapeutic release. A microfluidic capillary device was developed to synthesize polymer-based nanoparticles (polymersomes) while maintaining coacervate phase separation. This method allows for uniform polymersome synthesis while simultaneously encapsulating coacervates (FIG. 16).

To assemble the microcapillary-based microfluidic device, two PEEK chromatography tees were connected by a square capillary to ensure concentric alignment of two tapered round capillaries defining the breakup zone; the capillaries were sealed using PVC tubing and flangeless ferrules (See Benson, B. R., Stone, H. A. & Prud'homme, R. K. An “Off-the-shelf” Capillary Microfluidic Device that Enables Tuning of the Droplet Breakup Regime at Constant Flow Rates. Lab Chip 13, 4507-4511 (2013)). The oil phase, a 38:62 chloroform solution of Polyethylene glycol-b-polylactic acid (PEG-PLA) (1 k, 5 k), and the aqueous phases (4.0 wt % polyvinyl alcohol (PVA) for the inner phase, 0.1 wt % PVA and 0.1 M NaCl for the outer) were injected into the device at 800, 700, and 3000 microliters/hour to form water/oil/water emulsions as polymersome synthesis templates (Martino, C., Kim, S. H., Horsfall, L., Abbaspourrad, A., Rosser, S. J., Cooper, J. & Weitz, D. A. Protein expression, aggregation, and triggered release from polymersomes as artificial cell-like structures. Angew. Chemie—Int. Ed. 51, 6416-6420 (2012)). Polymersomes were separated by centrifugation and analyzed for size distribution and Z-average diameter on a Malvern Zetasizer Nano ZS via dynamic light scattering. Some samples were stained with uranyl acetate at imaged via Transmission Electron Microscopy using an HT7830 microscope. Results indicate that the microfluidic device successfully generated monodisperse nanosized polymersomes while potentially maintaining coacervate phase separation within water/oil/water double emulsion templates.

### Example 4

The following example demonstrates the ROS-triggered release of RNA from redox-responsive coacervates.

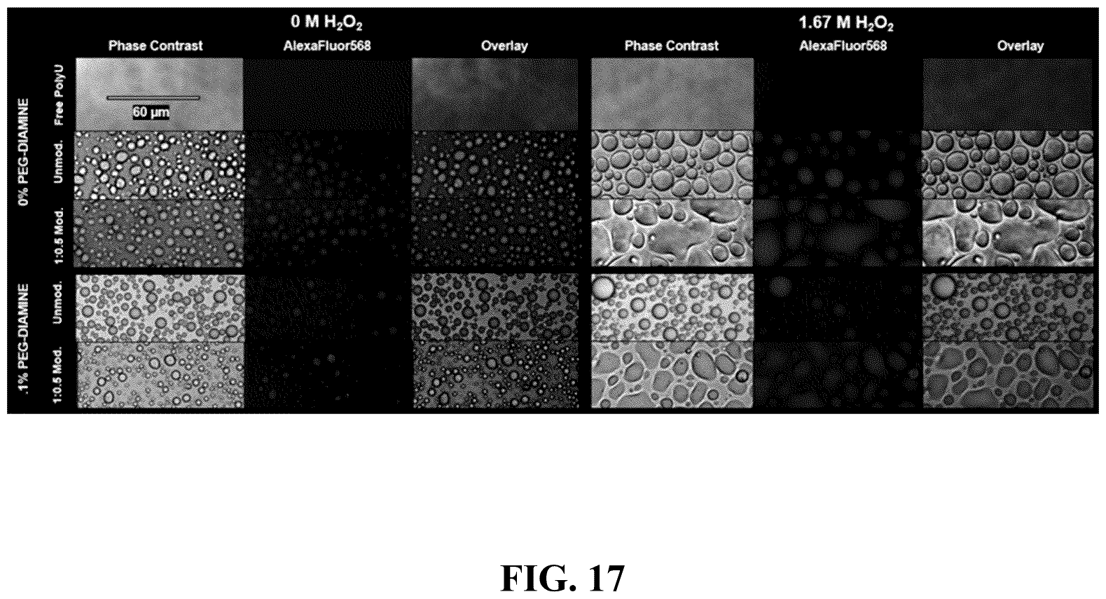

ROS-triggered release of fluorescently labeled RNA from redox-responsive coacervates: Coacervates were formed by mixing spermine and polyU in the presence of 2% AlexaFluor568-labeled 20-mer polyU to visualize RNA localization. Phase contrast, Texas Red fluorescence (AlexaFluor568), and overlay images are shown in FIG. 17 for: (1) free RNA (no spermine), (2) unmodified spermine/polyU coacervates, and (3) 1:0.5 DTSSP-modified spermine/polyU coacervates, both before and after treatment with 1.67 M H₂O₂ for 15 minutes. Unmodified coacervates retained their phase-separated morphology and internal fluorescence signal post-treatment. In contrast, the modified coacervates lost their circular morphology and red fluorescence signal upon oxidation, indicating dissolution of the coacervate phase and concurrent release of RNA, confirming that RNA is sequestered within the condensed phase and released upon oxidative trigger.

Quantitative analysis of turbidity and fluorescence before and after H₂O₂ treatment: Turbidity (absorbance at 500 nm) and total AlexaFluor568 fluorescence were measured for unmodified and 1:0.5 modified spermine/polyU coacervates before and after treatment with 1.67 M H₂02. As detailed in Table 2, unmodified samples showed minimal change in turbidity and a decrease in fluorescence upon coacervation, consistent with fluorescence quenching of RNA within the dense phase. In contrast, modified samples showed a decrease in turbidity and an increase in fluorescence upon H₂O₂ treatment, consistent with coacervate dissolution and RNA release into the dilute phase. Together, these results confirm that RNA is sequestered within the coacervate and released upon oxidative stimulus in redox-responsive systems.

TABLE 2
Quantitative analysis of turbidity and fluorescence
before and after H₂O₂ treatment

| | ABSORBANCE | | FLUORESCENCE | |
|---|---|---|---|---|
| | Before H₂O₂ | After H₂0₂ | Before H₂O₂ | After H₂0₂ |
| Unmodified | 94.72 +/− | −4.93 +/− | 73.54 +/− | −7.40 +/− |
| | 0.14 | 0.28 | 2.70 | 9.18 |
| Modified | 95.24 +/− | −7.17 +/− | 75.44 +/− | +4.07 +/− |
| | 0.03 | 1.97 | 4.26 | 6.18 |

### Example 5

The following example demonstrates that adding low concentrations of PEG-diamine enhances coacervate formation and stability in CHO cell culture media. The example also demonstrates that ROS-induced coacervate dissolution can be seen by eye, demonstrating a novel composition and method for use as a visual, label-free ROS sensor for oxidative stress monitoring in biomanufacturing. The materials are engineered to respond to reactive oxygen species (ROS) and can be applied in biomanufacturing, including cell culture monitoring and control, particularly for Chinese Hamster Ovary (CHO) cells and other industrially relevant cell lines.

In biomanufacturing, elevated ROS levels can indicate metabolic imbalance, stress responses, or impending cell death, particularly in sensitive mammalian expression systems such as CHO cells. Continuous monitoring of ROS in these systems is essential for maintaining product yield and quality. Current ROS assays are largely destructive or require external reagents, which limit their integration into real-time feedback loops. There remains an unmet need for materials that can sense and respond to ROS in situ, particularly in non-destructive, biocompatible formats compatible with live cell environments and scalable for industrial use.

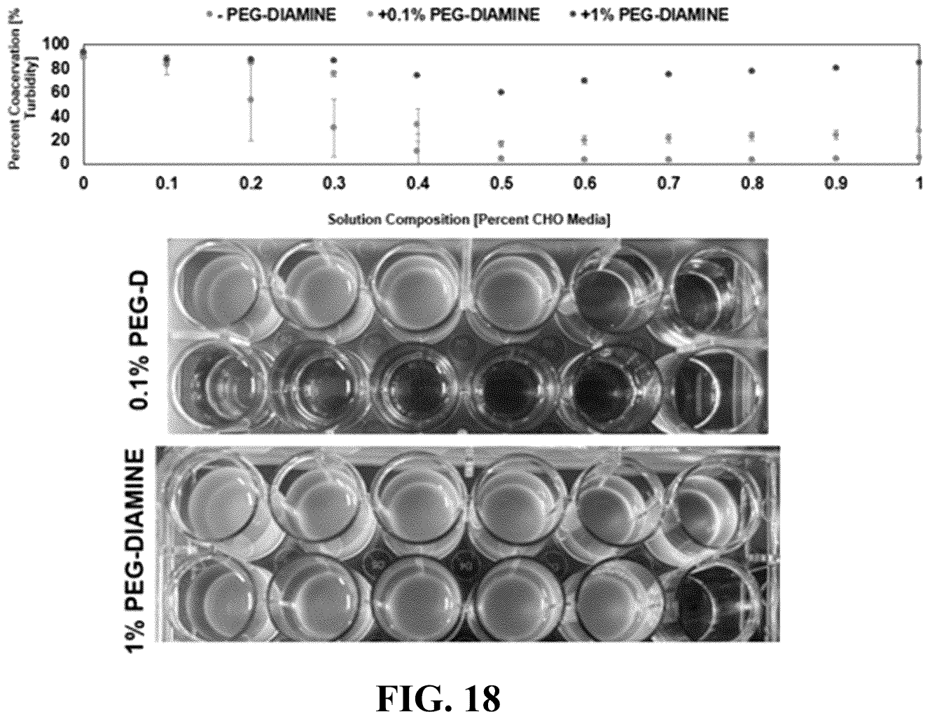

Optimization of redox-responsive coacervate formation in CHO cell culture media using PEG-diamine. FIG. 18 shows the quantification of coacervate formation across increasing ratios of CHO media to buffer, showing improved tolerance and stability in media-containing environments upon addition of PEG-diamine. Coacervates were formed using unmodified or modified spermine/polyU structures with 0%, 0.1%, or 1.0% PEG-diamine (w/w). The wells correspond to the data points in the chart in FIG. 18: well A1 is x=0 (100% coacervate buffer, 0% CHO media) and well B5 is x=1 (100% CHO media). The last well in row B on both plates is empty. The dip in percent coacervation in the scatter plot should correspond to the dip in cloudiness in the well plates. However, beyond A5 for the 1% PEG-diamine samples, the observed cloudiness is due to precipitation rather than liquid-liquid coacervation. The threshold CHO media concentration compatible with coacervate formation increased substantially with PEG-diamine incorporation, indicating enhanced robustness of the system in cell culture-relevant conditions. Photographs of 24-well plates visually demonstrate coacervate turbidity under ambient light, highlighting the ability to observe phase separation and dissolution by eye. Wells containing higher PEG-diamine concentrations show increased turbidity across a wider range of media conditions, consistent with enhanced coacervate stability.

Conceptual schematic of the coacervate-based sensing workflow: an aliquot of CHO culture media is added to pre-formed redox-responsive coacervates. In oxidative environments, elevated ROS levels (e.g., hydrogen peroxide) induce coacervate dissolution, releasing encapsulated RNA and leading to a visually detectable decrease in turbidity and/or change in colorimetric or fluorescent signal. This platform enables rapid, label-free assessment of oxidative stress states in biomanufacturing workflows.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A composition for ribonucleic acid (RNA)-based therapy, said composition comprising a complex coacervate of RNA and a cationic polymer.

2. The composition of claim 1 further comprising a nanoparticle encasing the complex coacervate.

3. The composition of claim 1, wherein the cationic polymer is a spermine or a spermidine.

4. The composition of claim 1, wherein the cationic polymer is spermine and is modified with DTSSP or dithiobis(succinimidyl propionate) (DSP/DTSP).

5. The composition of claim 4, wherein the spermine is modified with DTSSP at a ratio between 1:0 and 1:1 spermine/DTSSP.

6. The composition of claim 1, wherein the RNA is modified RNA.

7. The composition of claim 1, wherein the RNA is mRNA, siRNA, miRNA, lncRNA, shRNA, crRNA, gRNA, rRNA, or tRNA.

8. The composition of claim 1, wherein the nanoparticle has a diameter between 100-200 nm.

9. The composition of claim 1, further comprising a stabilizing agent.

10. The composition of claim 9, wherein the stabilizing agent is polyethylene glycol (PEG) or PEG-diamine.

11. A method of delivering RNA to a cytosol of a cell, the method comprising:
modifying a cationic composition with DTSSP or DTSP;
adding the cationic composition to an RNA composition to form a cationic polymer/RNA structure;
encapsulating the cationic polymer/RNA structure in a nanoparticle;
contacting the nanoparticle to a cell containing cytosol, the cell optionally located in an area of heightened reactive oxygen species (ROS) levels; and
optionally increasing a reactive oxygen species (ROS) level of the cytosol to modulate a separation of the cationic polymer and the RNA.

12. The method of claim 11 wherein the cationic polymer is spermine or spermidine.

13. The method of claim 11 wherein the ROS level is increased by a disease, inflammation, or providing ionizing radiation.

14. The method of claim 11 wherein the nanoparticle has a diameter between 100-200 nm.

15. The method of claim 11 wherein the RNA is mRNA, siRNA, miRNA, lncRNA, shRNA, crRNA, gRNA, rRNA, or tRNA.

16. The method of claim 11 wherein the separation is monitored by measuring the light absorbance (turbidity), by measuring fluorescence, or by measuring a radiolabel.

17. A method of providing RNA therapy to a patient, the method comprising:
modifying a spermine composition with DTSSP or DTSP;
adding the spermine to a RNA composition to form a spermine/RNA structure;
encapsulating the RNA/spermine structure in a nanoparticle;
delivering the nanoparticle to a region of therapy in a patient; and optionally exposing the region of therapy to ionizing radiation.

18. The method of claim 16 wherein the nanoparticle is delivered to the region of therapy via injection or intravascular administration.

19. The method of claim 16 wherein the nanoparticle has a diameter between 100-200 nm.

20. The method of claim 16 wherein the RNA is delivered to the cytosol for therapeutic applications.

21. The method of claim 16 wherein the RNA is labeled.
